(19) United States
(12) Patent Application Publication
(10) Pub. No.: US 2026/0108310 A1
(43) Pub. Date: Apr. 23, 2026

(54) KEY FRAME IDENTIFICATION FOR POST PERCUTANEOUS INTERVENTION INTRAVASCULAR IMAGING BASED ON STENT LOCATIONS

(71) Applicant: Boston Scientific Scimed, Inc., Maple Grove, MN (US)

(72) Inventors: Viola Kim Holman Narveson, Crystal, MN (US); Achala Thippeswamy, San Francisco, CA (US)

(73) Assignee: Boston Scientific Scimed, Inc., Maple Grove, MN (US)

(21) Appl. No.: 19/365,715

(22) Filed: Oct. 22, 2025

Related U.S. Application Data
(60) Provisional application No. 63/710,510, filed on Oct. 22, 2024.

(51) Int. Cl. A61B 34/20; A61B 8/08; A61B 8/12

(52) U.S. Cl. CPC A61B 34/20; A61B 8/0891; A61B 8/12; A61B 2034/2063; A61B 2034/2065

(57) ABSTRACT

The present disclosure provides to process intravascular images, such as intravascular ultrasound (IVUS) image frames, to identify key frames related to a stent represented in the images. A distal and a proximal key frame can be identified from a series of intravascular image frames based on a lumen area (e.g., raw, smoothed, etc.) and plaque burden as well as based on a threshold distance from the distal and/or proximal stent edges and identified side branches.

### CROSS REFERENCE TO RELATED APPLICATIONS

This application claims the benefit of priority under 35 U.S.C. § 119 to U.S. Provisional Ser. No. 63/710,510, filed Oct. 22, 2024, each of which is herein incorporated by reference in its entirety.

### TECHNICAL FIELD

The present disclosure generally relates to intravascular imaging devices and systems arranged to capture a series of image frames and to graphical user interfaces to display indications of the captured image frames. Particularly, but not exclusively, the present disclosure relates to identifying key frames from the captured image frames based in part on the location of a stent in the intravascular image frames.

### BACKGROUND

Intravascular imaging (IVI) devices are insertable into a patient's vasculature and configured to capture images from within the vessel lumen. Two common intravascular imaging modalities are intravascular ultrasound (IVUS) and optical coherence tomography (OCT). Such imaging modalities have proven diagnostic capabilities for a variety of diseases and disorders. For example, IVI devices are often used as part of a percutaneous coronary intervention (PCI) and can be used to plan, treat, and assess treatment for various obstructive coronary artery diseases, such as, unstable angina, acute myocardial infarction (MI), coronary artery disease (CAD), or the like.

As noted, an example IVI system is an IVUS system. An IVUS system includes a control module with a pulse generator, a motor drive unit, image acquisition and processing components, and a monitor. The IVUS system further includes a catheter with an ultrasound transducer included as part of the distal end of the catheter. The catheter is positioned in a lumen or cavity within, or in proximity to, a region to be imaged, such as a cardiac vessel. Often, a series of images or series of image frames are captured while the catheter is moved (e.g., pulled proximally, or the like) within the vessel lumen.

This series of image frames represent the vessel lumen structure (e.g., vessel wall, lumen, plaque, stents, etc.) along a longitudinal section of the vessel. Such images can be captured as part of a pre-PCI procedure to aid a physician in determining how to treat the patient, for example, what stent size is appropriate to treat a stenosis, stent landing zones, or the like. Further, such images can be captured as part of a post-PCI procedure to assess the results of the procedure, such as the placement and/or expansion of the stent.

However, it can be difficult for physicians to visualize the complete structure of the vessel lumen and/or the effectiveness of the PCI from the raw series of image frames. For example, it is difficult for physicians to identify key locations within an IVUS run. These key locations are often used as the basis for calculations and/or determinations related to making clinical decisions as part of a post-PCI procedure.

Thus, there here is a need for IVI systems and methods to process, annotate, visualize, and/or display images captured by IVI systems as part of a post-PCI procedure to assist physicians in making clinical decisions. Particularly, there is a need for automatically identifying key frames in the IVI data related to a stent.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In general, the present disclosure provides systems and techniques to identify key frames from a series of image frames captured via an IVI modality as part of a post-PCI procedure. Specifically, the present disclosure provides systems and techniques to identify key frames from post-PCI IVI imaging modality runs that have a stent within the imaged region. The identified key frames can be indicated for a user (e.g., physician, or the like) via a graphical user interface (GUI). Further, the identified key frames can be used as a basis for deriving stent placement metrics (e.g., stent expansion, or the like) that allow users to quickly assess the stent placement and make clinical decisions to improve patient outcomes related to the post-PCI procedure.

Of note, the present disclosure provides systems and techniques to identify key frame markers (KFMs) for a stent or a stent segment from a series of image frames captured via an IVI imaging modality based in part on lumen area, plaque burden, detected stent edges, and detected side branches. The KFMs can include a distal key frame, a proximal key frame, and/or a minimum key frame. Further, the systems and techniques can select as a default reference frame either the distal key frame or the proximal key frame (e.g., if the stent is cut off on the distal end the in the image frames, or the like) and generate a GUI comprising indications of the series of image frames, the KFMs, and the reference frame.

In some embodiments, the disclosure can be implemented as a computer-implemented method for an intravascular image navigation system. The method can comprise receiving, at a processor, a series of intravascular image (IVI) frames captured along a section of a cardiac artery of a patient where a stent is disposed in the section of the cardiac artery; identifying, by the processor, an edge of the stent from the series of IVI frames; determining, by the processor, whether a location of the edge of the stent is less than or equal to a first threshold distance from an end of the section of the cardiac artery; and identifying, by the processor, a frame of the series of IVI frames captured at the end of the section of the cardiac artery as a key frame if the location of the edge of the stent is less than or equal to the first threshold distance from the end of the section of the cardiac artery; or identifying, by the processor, a frame of one or more first frames of the series of IVI frames as the key frame based on a lumen area and a plaque burden for each of the one or more first frames if the location of the edge of the stent is not less than or equal to the first threshold distance from the end of the section of the cardiac artery, wherein the one or more first frames are frames captured between the end of the section of the cardiac artery and the edge of the stent.

With further embodiments of the method, the edge of the stent is a distal edge, the end of the section of the cardiac artery is a distal end, the key frame is a distal key frame, and the method can comprise identifying, by the processor, an proximal edge of the stent from the series of IVI frames; determining, by the processor, whether a location of the proximal edge of the stent is less than or equal to a second threshold distance from a proximal end of the section of the cardiac artery; and identifying, by the processor, another frame of the series of IVI frames captured at the proximal end of the section of the cardiac artery as a proximal key frame if the location of the proximal edge of the stent is less than or equal to the second threshold distance from the proximal end of the section of the cardiac artery; or identifying, by the processor, another frame of one or more second frames of the series of IVI frames as the proximal key frame based on a lumen area and a plaque burden for each of the one or more second frames if the location of the proximal edge of the stent is not less than or equal to the second threshold distance from the proximal end of the section of the cardiac artery, wherein the one or more second frames are frames captured between the proximal end of the section of the cardiac artery and the proximal edge of the stent.

With further embodiments of the method, the first threshold distance and/or the second threshold distance are 0.5 millimeters (mm) or wherein the first threshold distance and/or the second threshold distance are between 0.25 mm and 0.75 mm.

With further embodiments, the method can comprise identifying, by the processor, a first subset of frames of the series of IVI frames based on an automatic stent detection process, wherein the first subset of frames comprises an indication of the stent.

With further embodiments, the method can comprise identifying, by the processor, the edge of the stent based on the first subset of frames.

With further embodiments, the method can comprise identifying, by the processor, a second subset of frames of the series of IVI frames based on an automatic side branch detection process, wherein the second subset of frames comprises an indication of one or more side branches.

With further embodiments, the method can comprise determining, by the processor, whether the one or more first frames of the series of IVI frames comprises indications of the one or more side branches based on the second subset of frames; and identifying, by the processor, the frame of the one or more first frames located distal to the location of the distal edge of the stent by the first threshold distance as the distal key frame if the one or more first frames of the series of IVI frames comprises indications of the one or more side branches.

With further embodiments, the method can comprise determining, by the processor, whether the one or more second frames of the series of IVI frames comprises indications of the one or more side branches based on the second subset of frames; and identifying, by the processor, the frame of the one or more second frames located proximal to the location of the proximal edge of the stent by the second threshold distance as the proximal key frame if the one or more second frames of the series of IVI frames comprises indications of the one or more side branches.

With further embodiments, the method can comprise deriving, by the processor, the lumen area and the plaque burden for each frame of the series of IVI frames based on a lumen assessment process.

With further embodiments of the method, the lumen area is a raw lumen area and the method can comprise deriving, by the processor, a smoothed lumen area from each of the raw lumen areas; determining, by the processor, whether any one of the one or more first frames have a plaque burden less than or equal to a plaque burden threshold; and determining, by the processor, whether any one of the one or more second frames have a plaque burden less than or equal to a plaque burden threshold; identifying, by the processor, the frame of the one or more first frames furthest from the distal edge of the stent as the distal key frame based on a determination that not one of the one or more first frames have a plaque burden less than or equal to the plaque burden threshold; or identifying, by the processor, the frame of the one or more first frames closest to the distal edge of the stent that has (i) the largest raw lumen area and the largest smoothed lumen area and (ii) a plaque burden less than or equal to the plaque burden threshold as the distal key frame; and identifying, by the processor, the frame of the one or more second frames furthest from the proximal edge of the stent as the proximal key frame based on a determination that not one of the one or more second frames have a plaque burden less than or equal to the plaque burden threshold; or identifying, by the processor, the frame of the one or more second frames closest to the proximal edge of the stent that has (i) the largest raw lumen area and the largest smoothed lumen area and (ii) a plaque burden less than or equal to the plaque burden threshold as the proximal key frame.

With further embodiments of the method, the plaque burden threshold is 50% or between 40% and 60%.

With further embodiments, the method can comprise generating, by the processor, one or more graphical indications representing the series of IVI frames, the distal key frame, and the proximal key frame; and sending, by the processor, one or more information elements to a display to cause the display to display the one or more graphical indications as part of a graphical user interface.

With further embodiments of the method, the series of IVI frames and intravascular ultrasound (IVUS) frames.

In some embodiments, the disclosure can be implemented as apparatus, comprising a processor coupled to a memory, the memory comprising instructions executable by the processor, the processor configured to couple to an intravascular imaging (IVI) system and configured to execute the instructions, which instructions when executed cause the processor to implement any of the methods disclosure herein.

In some embodiments, disclosure can be implemented as at least one machine readable storage device, comprising a plurality of instructions that in response to being executed by a processor of an intravascular imaging (IVI) system cause the processor to implement any of the methods disclosed herein.

In some embodiments, the disclosure can be implemented as an intravascular image navigation system. The intravascular image navigation system can comprise a processor; and a memory storage device coupled to the processor, the memory storage device comprising instructions executable by the processor, which instructions when executed cause the intravascular image navigation system to receive a series of intravascular image (IVI) frames captured along a section of a cardiac artery of a patient where a stent is disposed in the section of the cardiac artery; identify an edge of the stent from the series of IVI frames; determine whether a location of the edge of the stent is less than or equal to a first threshold distance from an end of the section of the cardiac artery; and identify a frame of the series of IVI frames captured at the end of the section of the cardiac artery as a key frame if the location of the edge of the stent is less than or equal to the first threshold distance from the end of the section of the cardiac artery; or identify a frame of one or more first frames of the series of IVI frames as the key frame based on a lumen area and a plaque burden for each of the one or more first frames if the location of the edge of the stent is not less than or equal to the first threshold distance from the end of the section of the cardiac artery, wherein the one or more first frames are frames captured between the end of the section of the cardiac artery and the edge of the stent.

With further embodiments of the intravascular image navigation system, the edge of the stent is a distal edge, the end of the section of the cardiac artery is a distal end, the key frame is a distal key frame, and the memory storage device can further comprise instructions executable by the processor, which instructions when executed cause the intravascular image navigation system to identify a proximal edge of the stent from the series of IVI frames; determine whether a location of the proximal edge of the stent is less than or equal to a second threshold distance from a proximal end of the section of the cardiac artery; and identify another frame of the series of IVI frames captured at the proximal end of the section of the cardiac artery as a proximal key frame if the location of the proximal edge of the stent is less than or equal to the second threshold distance from the proximal end of the section of the cardiac artery; or identify another frame of one or more second frames of the series of IVI frames as the proximal key frame based on a lumen area and a plaque burden for each of the one or more second frames if the location of the proximal edge of the stent is not less than or equal to the second threshold distance from the proximal end of the section of the cardiac artery, wherein the one or more second frames are frames captured between the proximal end of the section of the cardiac artery and the proximal edge of the stent.

With further embodiments of the intravascular image navigation system, the first threshold distance and/or the second threshold distance are 0.5 millimeters (mm) or wherein the first threshold distance and/or the second threshold distance are between 0.25 mm and 0.75 mm.

With further embodiments of the intravascular image navigation system, the memory storage device can further comprise instructions executable by the processor, which instructions when executed cause the intravascular image navigation system to identify a first subset of frames of the series of IVI frames based on an automatic stent detection process, wherein the first subset of frames comprises an indication of the stent.

With further embodiments of the intravascular image navigation system, the memory storage device can further comprise instructions executable by the processor, which instructions when executed cause the intravascular image navigation system to identify the edge of the stent based on the first subset of frames.

With further embodiments of the intravascular image navigation system, the memory storage device can further comprise instructions executable by the processor, which instructions when executed cause the intravascular image navigation system to identify a second subset of frames of the series of IVI frames based on an automatic side branch detection process, wherein the second subset of frames comprises an indication of one or more side branches.

With further embodiments of the intravascular image navigation system, the memory storage device can further comprise instructions executable by the processor, which instructions when executed cause the intravascular image navigation system to determine whether the one or more first frames of the series of IVI frames comprises indications of the one or more side branches based on the second subset of frames; and identify the frame of the one or more first frames located distal to the location of the distal edge of the stent by the first threshold distance as the distal key frame if the one or more first frames of the series of IVI frames comprises indications of the one or more side branches.

With further embodiments of the intravascular image navigation system, the memory storage device can further comprise instructions executable by the processor, which instructions when executed cause the intravascular image navigation system to determine whether the one or more second frames of the series of IVI frames comprises indications of the one or more side branches based on the second subset of frames; and identify the frame of the one or more second frames located proximal to the location of the proximal edge of the stent by the second threshold distance as the proximal key frame if the one or more second frames of the series of IVI frames comprises indications of the one or more side branches.

With further embodiments of the intravascular image navigation system, the memory storage device can further comprise instructions executable by the processor, which instructions when executed cause the intravascular image navigation system to derive the lumen area and the plaque burden for each frame of the series of IVI frames based on a lumen assessment process.

With further embodiments of the intravascular image navigation system, the series of IVI frames and intravascular ultrasound (IVUS) frames.

In some embodiments, the disclosure can be implemented as at least one non-transitory machine readable storage devices, comprising instructions that in response to being executed by a processor of an intravascular imaging (IVI) system cause the IVI system to receive a series of intravascular image (IVI) frames captured along a section of a cardiac artery of a patient where a stent is disposed in the section of the cardiac artery; identify an edge of the stent from the series of IVI frames; determine whether a location of the edge of the stent is less than or equal to a first threshold distance from an end of the section of the cardiac artery; and identify a frame of the series of IVI frames captured at the end of the section of the cardiac artery as a key frame if the location of the edge of the stent is less than or equal to the first threshold distance from the end of the section of the cardiac artery; or identify a frame of one or more first frames of the series of IVI frames as the key frame based on a lumen area and a plaque burden for each of the one or more first frames if the location of the edge of the stent is not less than or equal to the first threshold distance from the end of the section of the cardiac artery, wherein the one or more first frames are frames captured between the end of the section of the cardiac artery and the edge of the stent.

With further embodiments of the at least one non-transitory machine readable storage devices, the edge of the stent is a distal edge, the end of the section of the cardiac artery is a distal end, the key frame is a distal key frame, and the storage devices can comprise instructions that in response to being executed by the processor of the IVI system cause the IVI system to identify a proximal edge of the stent from the series of IVI frames; determine whether a location of the proximal edge of the stent is less than or equal to a second threshold distance from a proximal end of the section of the cardiac artery; and identify another frame of the series of IVI frames captured at the proximal end of the section of the cardiac artery as a proximal key frame if the location of the proximal edge of the stent is less than or equal to the second threshold distance from the proximal end of the section of the cardiac artery; or identify another frame of one or more second frames of the series of IVI frames as the proximal key frame based on a lumen area and a plaque burden for each of the one or more second frames if the location of the proximal edge of the stent is not less than or equal to the second threshold distance from the proximal end of the section of the cardiac artery, wherein the one or more second frames are frames captured between the proximal end of the section of the cardiac artery and the proximal edge of the stent.

With further embodiments of the at least one non-transitory machine readable storage devices, the storage device can further comprise instructions that in response to being executed by the processor of the IVI system cause the IVI system to derive the lumen area and the plaque burden for each frame of the series of IVI frames based on a lumen assessment process.

With further embodiments of the at least one non-transitory machine readable storage devices, the lumen area is a raw lumen area and the storage device can further comprise instructions that in response to being executed by the processor of the IVI system cause the IVI system to derive a smoothed lumen area from each of the raw lumen areas; determine whether any one of the one or more first frames have a plaque burden less than or equal to a plaque burden threshold; and determine whether any one of the one or more second frames have a plaque burden less than or equal to a plaque burden threshold; identify the frame of the one or more first frames furthest from the distal edge of the stent as the distal key frame based on a determination that not one of the one or more first frames have a plaque burden less than or equal to the plaque burden threshold; or identify the frame of the one or more first frames closest to the distal edge of the stent that has (i) the largest raw lumen area and the largest smoothed lumen area and (ii) a plaque burden less than or equal to the plaque burden threshold as the distal key frame; and identify the frame of the one or more second frames furthest from the proximal edge of the stent as the proximal key frame based on a determination that not one of the one or more second frames have a plaque burden less than or equal to the plaque burden threshold; or identify the frame of the one or more second frames closest to the proximal edge of the stent that has (i) the largest raw lumen area and the largest smoothed lumen area and (ii) a plaque burden less than or equal to the plaque burden threshold as the proximal key frame.

With further embodiments of the at least one non-transitory machine readable storage devices, the plaque burden threshold is 50% or between 40% and 60%.

With further embodiments of the at least one non-transitory machine readable storage devices, the storage devices can further comprise instructions that in response to being executed by the processor of the IVI system cause the IVI system to generate one or more graphical indications representing the series of IVI frames, the distal key frame, and the proximal key frame; and send one or more information elements to a display to cause the display to display the one or more graphical indications as part of a graphical user interface.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A illustrates an example cardiac vasculature structure with a stent placed in one of the cardiac arteries and an IVUS catheter inserted into the one of the cardiac arteries.

FIG. 1B illustrates an IVUS imaging system including the IVUS catheter shown in FIG. 1A.

FIG. 2A illustrates an example series of IVUS image frames.

FIG. 2B illustrates a longitudinal representation of the example series of IVUS image frames of FIG. 2A.

FIG. 3 illustrates an IVUS navigation system.

FIG. 4 illustrates a logic flow to identify key frames for a post-PCI procedure.

FIG. 5 illustrates an image of a frame of a series of IVUS image frames.

FIG. 6 illustrates a graphical user interface.

FIG. 7 illustrates another graphical user interface.

FIG. 8 illustrates a logic flow to identify a distal key frame for a post-PCI procedure.

FIG. 9 illustrates a logic flow to identify a distal key frame for a post-PCI procedure.

FIG. 10 illustrates a computer-readable storage medium.

FIG. 11 illustrates a diagrammatic representation of a machine.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and operation, together with further objects and advantages will be better understood from the following description when considered in connection with the accompanying figures. It is to be expressly understood, however, that each of the figures is provided for the purpose of illustration and description and is not intended as a definition of the limits of the present disclosure.

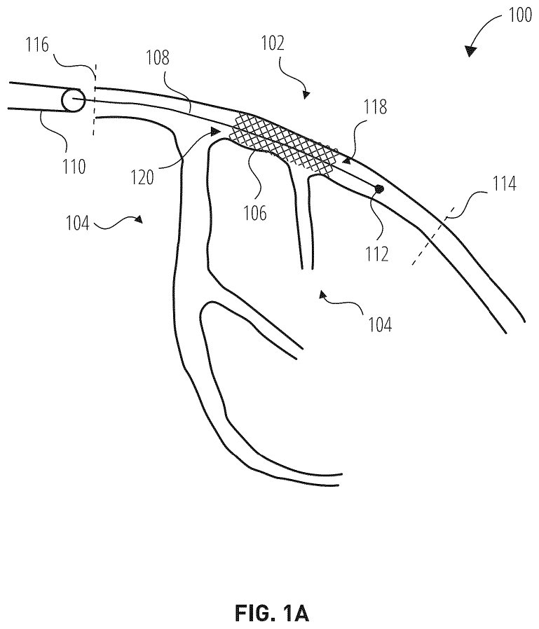

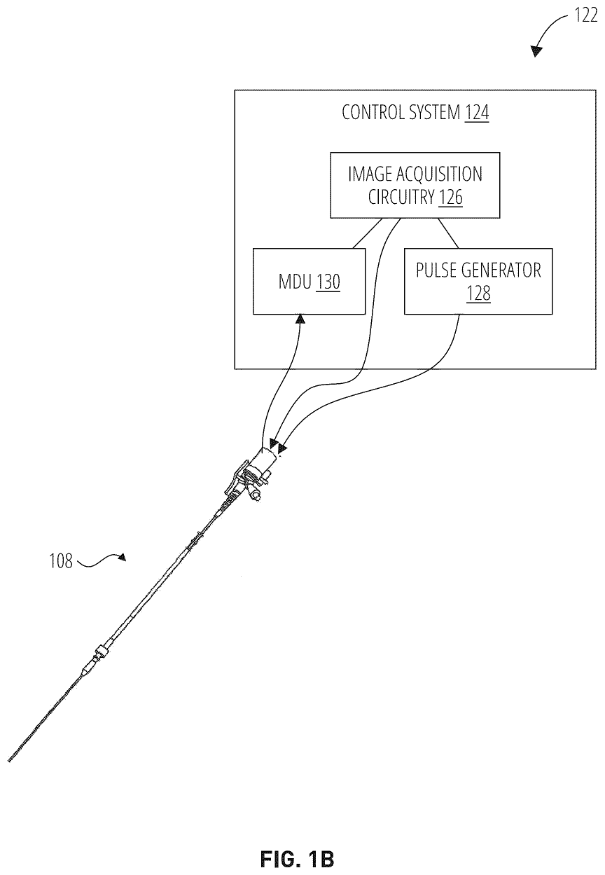

As outlined above, after some PCI procedures where a stent is placed in a section of a patient's vasculature, a post-PCI procedure can be performed. The post-PCI procedure is typically performed so that physicians can assess the stent placement, stent expansion, and/or other characteristics of the PCI procedure. FIG. 1A illustrates a cardiac vasculature structure 100 having a cardiac artery 102 (e.g., cardiac artery, vessel, or the like) with several side branches 104 (e.g., descending arteries, marginal arteries, vessels, or the like) branching off the cardiac artery 102. The cardiac vasculature structure 100 further depicts a stent 106 disposed (or placed) in a portion of the cardiac artery 102. It will be appreciated that the stent 106 may be placed after and/or as part of a PCI procedure to treat a stenosis. That is, stent 106 can be placed as part of a PCI procedure (e.g., stenting with balloon dilation, stenting with atherectomy, or the like) intended to dilate or open blocked or partially occluded vessels. In some examples, the stent 106 can be drug-eluting.

As noted above, PCI procedures can be guided by intravascular imaging. For example, prior to performing a PCI procedure, during a PCI procedure, and/or after a PCI procedure; an intravascular imaging catheter (e.g., IVUS imaging catheter 108) is inserted into the cardiac artery 102 via a guide catheter 110 and images of the cardiac artery 102 are captured. The IVUS imaging catheter 108 includes an ultrasound transducer 112 disposed on a distal end of the IVUS imaging catheter 108. During the post-PCI procedure, the IVUS imaging catheter 108 can be pulled back from a distal point 114 in the cardiac artery 102 to a proximal point 116 in the cardiac artery 102 and intravascular image frames are captured while the IVUS imaging catheter 108 is pulled back.

Where the PCI procedure involves placing the stent 106 in cardiac artery 102, a post-PCI procedure could involve imaging the cardiac artery 102 from the distal point 114 to the proximal point 116 to capture images of the stent 106. Physicians often use post-PCI procedures to assess how well the stent 106 is placed and/or how well the cardiac artery 102 stenosis is corrected relative to pre-PCI images. To assist physicians in assessing the placement of stent 106 in cardiac artery 102, the present disclosure provides to identify key frames from the IVUS image frames captured while the IVUS imaging catheter 108 is pulled back from distal point 114 to proximal point 116. This is explained in greater detail below. However, in general key frames are identified relative to the stent distal edge 118 and the stent proximal edge 120.

From the captured intravascular image frames and the identified key frames, various characteristics of the placement of stent 106 in cardiac artery 102 can be determined and a physician can use this determined information to assess the quality and/or effectiveness of the placement of stent 106 in cardiac artery 102.

It is noted that some patients may have multiple stents placed in a cardiac artery 102. For purpose of this disclosure, it is assumed that there is a single stent 106, or that one of multiple stents 106 is selected for identification of key frames as outlined herein. In some cases, where multiple stents 106 or stent segments are present, the most proximal stent can be selected by default.

The present disclosure can be implemented for any intravascular imaging modality. However, the balance of the disclosure uses IVUS imaging (e.g., captured via IVUS imaging catheter 108, or the like) as an exemplary modality. This is done for ease of discussion only. To aid this discussion, FIG. 1B illustrates an IVUS imaging system 122. The IVUS imaging system 122 is depicted with IVUS imaging catheter 108, which is couplable to a control system 124. The control system 124 may include, for example, an image acquisition circuitry 126, a pulse generator 128, and a motor drive unit (MDU) 130. In at least some embodiments, the pulse generator 128 forms electric pulses that may be input to one or more transducers (e.g., ultrasound transducer 112, or the like) disposed on IVUS imaging catheter 108.

With some embodiments, ultrasound transducer 112 can be part of an imaging core and coupled to the proximal end of IVUS imaging catheter 108 via a drive cable or drive shaft. Mechanical energy from the motor drive unit 130 can be used to rotate the imaging core (and thus the ultrasound transducer 112).

The electrical pulses generated by pulse generator 128 can be delivered to the ultrasound transducer 112 while the ultrasound transducer 112 is rotated by the motor drive unit 130. The electrical signals are transformed by the ultrasound transducer 112 into acoustic pulses that are transmitted through the tissue of cardiac vasculature structure 100. The tissue of cardiac vasculature structure 100 reflects the acoustic pulses, which are absorbed by the ultrasound transducer 112 and transformed into electric pulses. The transformed electric pulses are delivered to the image acquisition circuitry 126 and converted into IVUS images displayable on a monitor.

For example, image acquisition circuitry 126 can be configured to map scan line samples (e.g., radial scan line samples, or the like) to a two-dimensional Cartesian grid, which can be used as the basis for a series of IVUS images that can be displayed for a user. In at least some embodiments, the image acquisition circuitry 126 may also be used to control the functioning of one or more of the other components of the control system 124. For example, the image acquisition circuitry 126 may be used to control at least one of the frequency or duration of the electrical pulses transmitted from the pulse generator 128, the rotation rate of the imaging core by the motor drive unit 130, or the like. Additionally, where IVUS imaging system 122 is configured for automatic pullback, the motor drive unit 130 can control the velocity and/or length of the pullback region (e.g., distal point 114 to proximal point 116, or the like).

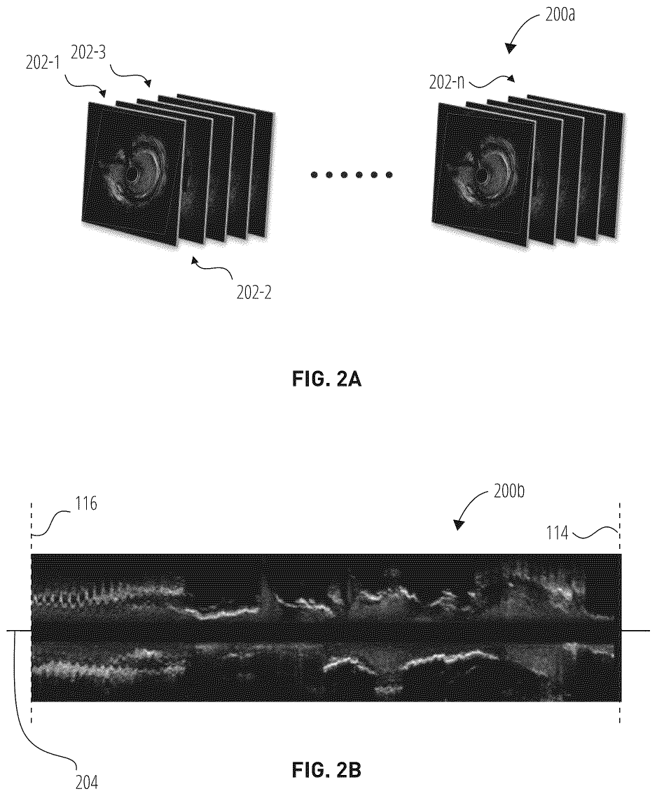

FIG. 2A and FIG. 2B illustrate a series of IVUS image frames 200a and a longitudinal view 200b of the series of IVUS image frames 200a. As described above, several IVUS image frames can be captured while IVUS imaging catheter 108 is moved through cardiac artery 102. For example, FIG. 2A depicts the series of IVUS image frames 200a including IVUS image frames 202-1, 202-2, 202-3 to 202-n, where n is a positive integer, in this case, greater than ten (10). In practice, n can be any positive integer greater than or equal to two (2) but will often be greater than one hundred.

The series of IVUS image frames 200a can be stacked or grouped and depicted longitudinally to represent a longitudinal slice of the cardiac artery 102 from distal point 114 to proximal point 116. For example, FIG. 2B illustrates longitudinal view 200b, which shows a longitudinal slice of cardiac artery 102 from distal point 114 to proximal point 116 formed from the series of IVUS image frames 200a.

As noted above, a stent (e.g., stent 106, or the like) may be represented in the series of IVUS image frames 200a (e.g., where they are captured as part of a post-PCI procedure, or the like). The disclosure provides to identify ones of the IVUS image frames 202-1, 202-2, 202-3 to 202-n as key frames based in part on the location of a stent in the series of IVUS image frames 200a. As used herein, the “location” of certain “features” (e.g., an edge of a stent such as the distal edge 118 or the proximal edge 120; a frame such as frame 202-1, 202-2, etc.; a side branch such as side branch 104; an end of a section of cardiac artery such as distal end 114 or proximal end 116, or the like) means the location of the respective feature along the length of the cardiac artery 102. These locations can be determined based on the series of IVUS image frames 200a. For example, where the series of IVUS image frames 200a are stacked or grouped as shown in FIG. 2B, a longitudinal axis 204 of the cardiac artery 102 represented by the series of IVUS image frames 200a can be determined. The longitudinal axis 204 can be representative of the path through the cardiac artery 102 in which the series of IVUS images 200a are captured. From the longitudinal axis 204 and the series of IVUS image frames 200a, the “location”of the “features”along the longitudinal axis 204 can be determined.

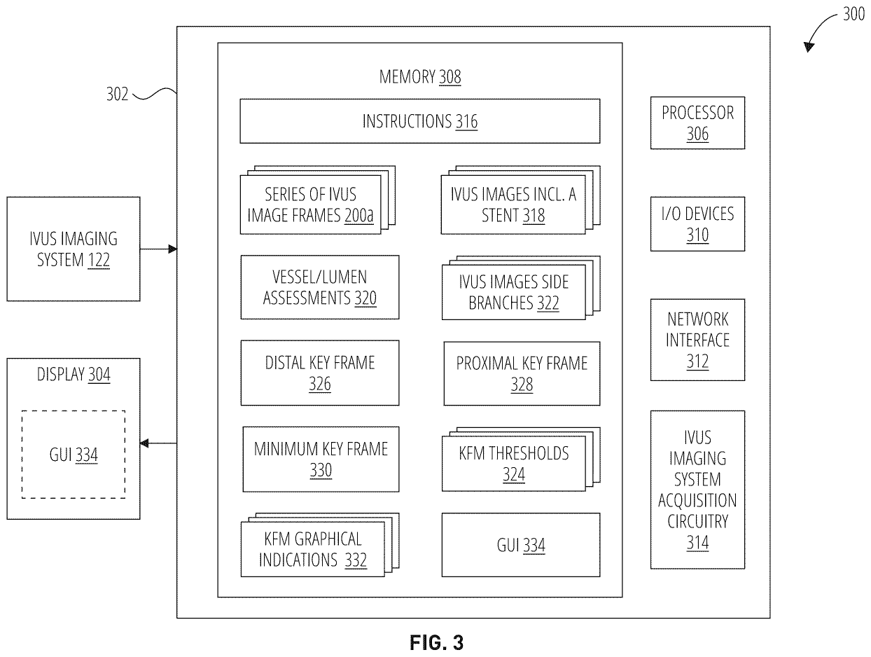

FIG. 3 illustrates an IVUS navigation system 300, which can be implemented to identify key frames as part of a post-PCI procedure to image a cardiac artery having a stent within the imaged region. In general, IVUS navigation system 300 is a system for processing, annotating, and/or presenting IVUS images. IVUS navigation system 300 can be implemented in a commercial IVUS guidance or navigation system, such as, for example, the AVVIGO™ Guidance System available from Boston Scientific®. The present disclosure provides advantages over prior or conventional IVUS navigation systems in that the automatic detection of key frames allows a user (e.g., physician, or the like) to interpret key calculations and make clinical decisions to improve patient outcomes related to the placement of the stent.

IVUS navigation system 300 includes a computing device 302 and display 304. Optionally, IVUS navigation system 300 includes an IVUS imaging system, such as IVUS imaging system 122. Where IVUS navigation system 300 includes IVUS imaging system 122, IVUS navigation system 300 could be implemented as part of control system 124 or alternatively, control system 124 could be implemented as part of computing device 302 of IVUS navigation system 300. IVUS navigation system 300 will be described with reference to cardiac vasculature structure 100 and IVUS imaging system 122 of FIG. 1A and FIG. 1B as well as series of IVUS image frames 200a and longitudinal view 200b of FIG. 2A and FIG. 2B for clarity and ease of discussion. However, it is noted that IVUS navigation system 300 could be implemented for use with another IVUS imaging system or even another intravascular imaging system utilizing a different imaging modality than IVUS imaging system 122.

Computing device 302 can be any of a variety of computing devices. In some embodiments, as noted above, computing device 302 can be incorporated into and/or implemented by a console to be coupled to an intravascular imaging device (e.g., IVUS imaging system 122, IVUS imaging catheter 108, or the like). With some embodiments, computing device 302 can be a workstation or server communicatively coupled to IVUS imaging system 122. With still other embodiments, computing device 302 can be provided by a cloud based computing device, such as, by a computing as a service system accessibly over a network (e.g., the Internet, an intranet, a wide area network, or the like). Computing device 302 can include processor 306, memory 308, input and/or output (I/O) devices 310, network interface 312, and IVUS imaging system acquisition circuitry 314.

Display 304 may include any of a variety of devices arranged to display graphical information, such as, a light emitting diode (LED) display, or the like. It is to be appreciated that although display 304 is depicted separate from computing device 302, display 304 could be implemented as part of computing device 302 or be distinct from computing device 302.

The processor 306 may include circuity or processor logic, such as, for example, any of a variety of commercial processors. In some examples, processor 306 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 306 may include graphics processing portions and may include dedicated memory, multiple-threaded processing and/or some other parallel processing capability. In some examples, the processor 306 may be an application specific integrated circuit (ASIC) or a field programmable integrated circuit (FPGA).

The memory 308 may include logic, a portion of which includes arrays of integrated circuits, forming non-volatile memory to persistently store data or a combination of non-volatile memory and volatile memory. It is to be appreciated, that the memory 308 may be based on any of a variety of technologies. In particular, the arrays of integrated circuits included in memory 308 may be arranged to form one or more types of memory, such as, for example, dynamic random access memory (DRAM), NAND memory, NOR memory, or the like.

I/O devices 310 can be any of a variety of devices to receive input and/or provide output. For example, I/O devices 310 can include, a keyboard, a mouse, a joystick, a foot pedal, a display, a touch enabled display, a haptic feedback device, an LED, or the like.

Network interface 312 can include logic and/or features to support a communication interface. For example, network interface 312 may include one or more interfaces that operate according to various communication protocols or standards to communicate over direct or network communication links. Direct communications may occur via use of communication protocols or standards described in one or more industry standards (including progenies and variants). For example, network interface 312 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 312 can include logic and/or features to enable communication over a variety of wired or wireless network standards. For example, network interface 312 may be arranged to support wired communication protocols or standards, such as, Ethernet, or the like. As another example, network interface 312 may be arranged to support wireless communication protocols or standards, such as, for example, Wi-Fi, Bluetooth, 5G, or the like.

The IVUS imaging system acquisition circuitry 314 may include circuity including custom manufactured or specially programmed circuitry configured to receive or receive and send signals with IVUS imaging system 122, including indications of intravascular images, intravascular image frames, or a series of intravascular image frames. For example, IVUS imaging system acquisition circuitry 314 can include image acquisition circuitry 126 and/or pulse generator 128.

Memory 308 can include instructions 316, series of IVUS image frames 200a, subset of IVUS image frames including a stent 318, vessel and/or lumen assessments 320, subset of IVUS image frames including a side branch 322, key frame marker thresholds 324, distal key frame 326, proximal key frame 328, minimum key frame 330, key frame marker graphical indications 332, and graphical user interface (GUI) 334.

During operation, processor 306 can execute instructions 316 to cause computing device 302 to receive (e.g., from IVUS imaging system 122, or the like) a recording of an “IVUS run” and store the recording as the series of IVUS image frames 200a in memory 308. For example, processor 306 can execute instructions 316 to receive information elements from IVUS imaging system 122 comprising indications of IVUS image frames 202-1, 202-2, 202-3 to 202-n, captured by IVUS imaging catheter 108 while IVUS imaging catheter 108 is pulled through cardiac artery 102 from distal point 114 to proximal point 116 through the stent 106. It is to be appreciated that the series of IVUS image frames 200a can be stored in a variety of image formats or even non-image formats or data structures.

The present disclosure provides to process the series of IVUS image frames 200a to identify key frames (e.g., the distal key frame 326, the proximal key frame 328, and/or the minimum key frame 330) from the frames of series of IVUS image frames 200a based in part on the location of the stent 106 in the series of IVUS image frames 200a. Accordingly, processor 306 can execute instructions 316 to identify a subset of frames of series of IVUS image frames 200a where a stent is represented. For example, processor 306 can execute instructions 316 to identify frames of series of IVUS image frames 200a (e.g., IVUS image frames 202-2, 202-3, etc.) where stent 106 is represented and store these frames, or indications of these frames, as subset of IVUS image frames including a stent 318. It is noted that in some embodiments, multiple stents 106 can be placed in cardiac artery 102. However, as outlined above, the present disclosure assumes that either there is one stent 106 or where multiple stents 106 are present, one of the multiple stents 106 is selected to be used as the basis for identifying key frames.

Complete details of stent detection techniques, or rather techniques to identify frames of the series of IVUS image frames 200a in which a stent is represented is beyond the scope of this disclosure. However, with some examples, processor 306 can execute instructions 316 to identify the subset of IVUS image frames including a stent 318 from series of IVUS image frames 200a using machine learning (ML) models trained to identify frames of a series of frames in which a stent is represented. As another example, processor 306 can execute instructions 316 to identify the subset of IVUS image frames including a stent 318 from the series of IVUS image frames 200a using image processing algorithms to identify stent features in frames of a series of frames. With yet another example, processor 306 can execute instructions 316 to identify the subset of IVUS image frames including a stent 318 from the series of IVUS image frames 200a using a combination of image processing techniques and ML models. For example, processor 306 can execute instructions 316 to apply a cross-sectional segmentation to the frames of the series of IVUS image frames 200a and then infer frames representing a stent from the frame segmentations using an ML model. It is noted that processor 306 can execute instructions 316 to identify edges of the stent or stents represented in series of IVUS image frames 200a. For example, processor 306 can execute instructions 316 to identify the frames (e.g., IVUS image frames 202-2, 202-3, etc.) where the edges of the stent 106 (e.g., the stent distal edge 118, the stent proximal edge 120, or the like).

Further, processor 306 can execute instructions 316 to derive vessel and/or lumen assessments 320 from the series of IVUS image frames 200a. In general, the vessel and/or lumen assessments 320 can include, for each frame of the series of IVUS image frames 200a, vessel and/or lumen borders, a raw vessel and/or lumen area, a smooth vessel and/or lumen area, a plaque burden, indications of a stent, indications of a stent edge, or the like. Complete details of derivation of vessel and/or lumen assessments is beyond the scope of this disclosure. However, with some examples, processor 306 can execute instructions 316 to determine assessments (e.g., boundaries, area, plaque burden, etc.) of the vessel and/or lumen depicted in each frame of the series of IVUS image frames 200a. With some embodiments, processor 306 can execute instructions 316 to determine the assessments using image processing techniques, ML models, or a combination of image processing techniques and ML models.

Additionally, processor 306 can execute instructions 316 to identify side branches from the series of IVUS image frames 200a. Complete details of side branch identification from IVUS image frames is beyond the scope of this disclosure. However, with some examples, processor 306 can execute instructions 316 to identify frames of the series of IVUS image frames 200a where side branches are represented and store these frames, or indications of these frames, as the subset of IVUS image frames including a side branch 322. For example, processor 306 can execute instructions 316 to identify the subset of IVUS image frames including a side branch 322 from the series of IVUS image frames 200a using image processing techniques, ML models, or a combination of image processing techniques and ML models.

Examples of processing IVUS images to detect stents, derive assessments, and/or identify side branches are described in more detail in United States Patent Application Publication No. 2023/0157672, titled “Intravascular Ultrasound Imaging and Calcium Detection Methods” and filed on Mar. 1, 2022; United States Patent Application Publication No. 2023/0112017, titled “Medical Device Systems for Automatic Lesion Assessment” and filed on Oct. 5, 2022; United States Patent Application Publication No. 2023/0380806, titled “Systems and Methods for Intravascular Visualization”and filed on May 26, 2023; United States Patent Application Publication No. 2024/0081781, titled “Graphical User Interface for Intravascular Ultrasound Stent Display” and filed on Sep. 13, 2023; which applications are each incorporated herein by reference in their entirety.

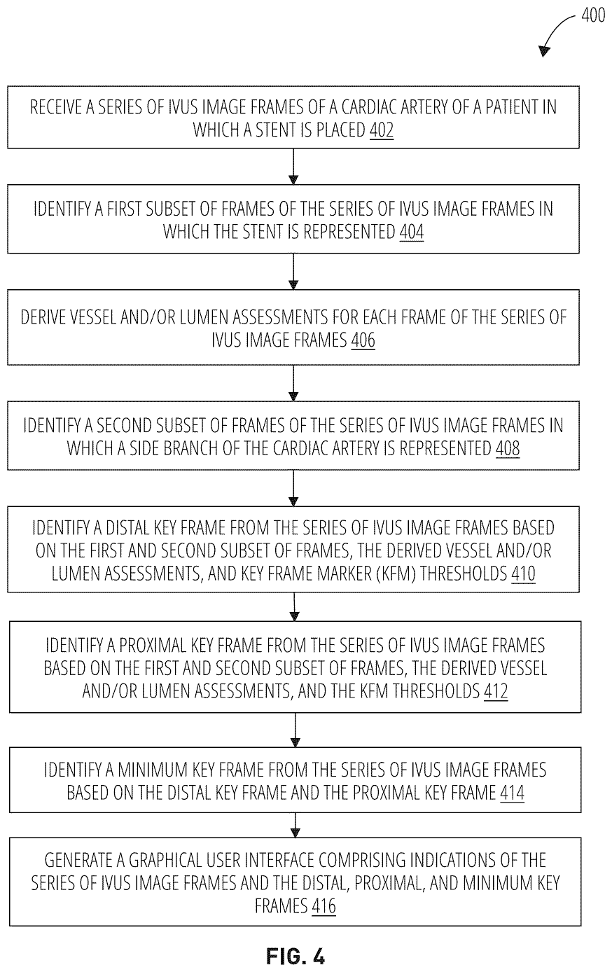

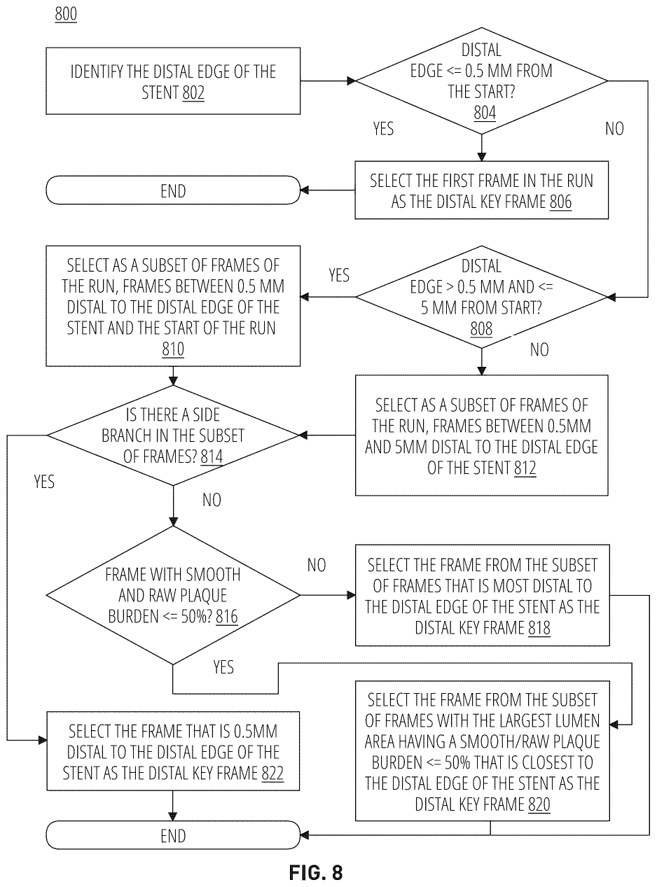

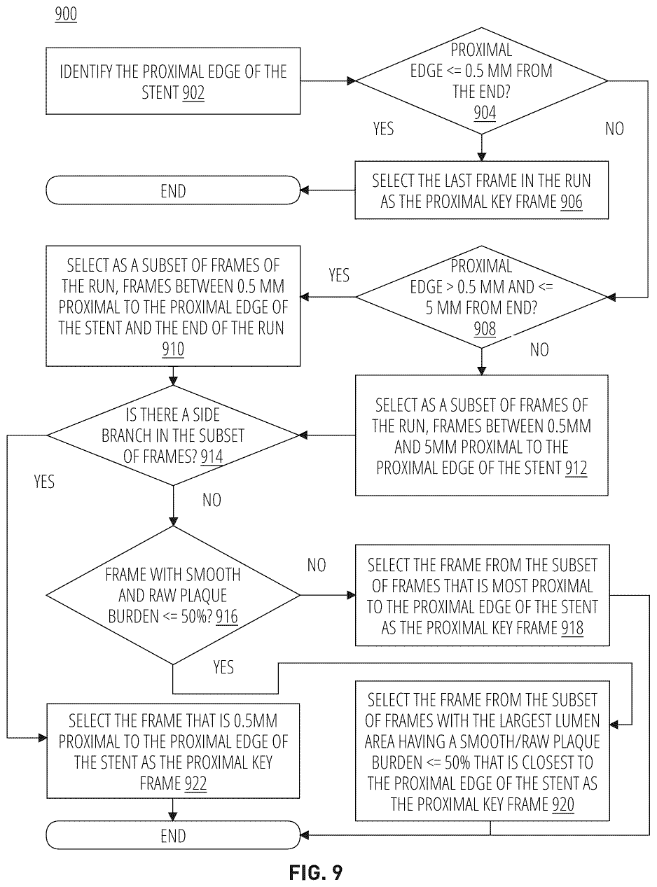

Processor 306 can execute instructions 316 to identify the key frames (e.g., the distal key frame 326, the proximal key frame 328, and/or the minimum key frame 330) from the series of IVUS image frames 200a based on the key frame marker thresholds 324 and subset of IVUS image frames including a stent 318, the subset of IVUS image frames including a side branch 322, and/or the vessel and/or lumen assessments 320. Examples of this are described in greater detail below, for example, with reference to logic flow 400 and FIG. 4, logic flow 800 of FIG. 8, and logic flow 900 of FIG. 9. However, as a general description, processor 306 can execute instructions 316 to identify the distal key frame 326 and the proximal key frame 328 as the frames of the series of IVUS image frames 200a having the largest lumen area with a plaque burden of less than or equal to a threshold value and which are within a threshold distance (e.g., key frame marker thresholds 324, or the like) from the stent edges and/or detected side branches.

Processor 306 can execute instructions 316 to identify the key frames (e.g., the distal key frame 326, the proximal key frame 328, and/or the minimum key frame 330) from the series of IVUS image frames 200a based on the key frame marker thresholds 324, the subset of IVUS image frames including a stent 318, the subset of IVUS image frames including a side branch 322, and/or the vessel and/or lumen assessments 320. Further, processor 306 can execute instructions 316 to generate key frame marker graphical indications 332 for the identified key frames (e.g., the distal key frame 326, the proximal key frame 328, and/or the minimum key frame 330) and/or generate the graphical user interface (GUI) 334 comprising graphical representations of the series of IVUS image frames 200a, the distal key frame 326, the proximal key frame 328, and/or the minimum key frame 330 as well as the key frame marker graphical indications 332 and cause GUI 334 to be displayed on display 304.

FIG. 4 illustrates a logic flow 400 to identify key frames from a series of IVUS image frames captured during a post-PCI procedure. The logic flow 400 can be implemented by IVUS navigation system 300 and will be described with reference to IVUS navigation system 300 for clarity of presentation. However, it is noted that logic flow 400 could also be implemented by an IVUS navigation system different than IVUS navigation system 300. Further, logic flow 400 is described with reference to cardiac vasculature structure 100 and IVUS imaging system 122 of FIG. 1A and FIG. 1B and the series of IVUS image frames 200a and longitudinal view 200b of FIG. 2A and FIG. 2B for clarity of presentation.

Logic flow 400 can begin at block 402. At block 402 “receive a series of intravascular ultrasound (IVUS) image frames of a vessel of a patient in which a number of stents are placed” a series of IVUS image frames captured during a post-PCI procedure via an IVUS catheter percutaneously inserted in a vessel of a patent can be received. For example, information elements comprising indications of series of IVUS image frames 200a can be received from IVUS imaging system 122 where IVUS imaging catheter 108 is (or was) percutaneously inserted into cardiac artery 102 of cardiac vasculature structure 100. As described above, the series of IVUS image frames 200a includes several frames (e.g., IVUS image frames 202-1, 202-2, and 202-3 to 202-n) captured while the IVUS imaging catheter 108 is pulled back from the distal point 114 to proximal point 116. Processor 306 can execute instructions 316 to receive information elements comprising indications of series of IVUS image frames 200a from IVUS imaging system 122, or directly from IVUS imaging catheter 108 as may be the case.

Continuing to block 404 “identify a first subset of frames of the series of IVUS image frames in which a stent is represented” a first subset of the frames of the series of IVUS image frames in which a stent is represented are identified. For example, processor 306 can execute instructions 316 to identify the subset of IVUS image frames including a stent 318 from the series of IVUS image frames 200a based on a stent detection technique and/or system.

Continuing to block 406 “derive vessel and/or lumen assessments for each frame of the series of IVUS image frames” vessel and/or lumen assessments for each frame of the series of IVUS image frames can be derived. For example, processor 306 can execute instructions 316 to derive vessel and/or lumen assessments 320 for frames of series of IVUS image frames 200a based on a vessel and/or lumen assessment technique and/or system. With some embodiments, processor 306 can execute instructions 316 to derive vessel and/or lumen assessments 320 for each frame of series of IVUS image frames 200a as stated above, while in other embodiments processor 306 can execute instructions 316 to derive vessel and/or lumen assessments 320 for each frame of the subset of IVUS image frames including a stent 318.

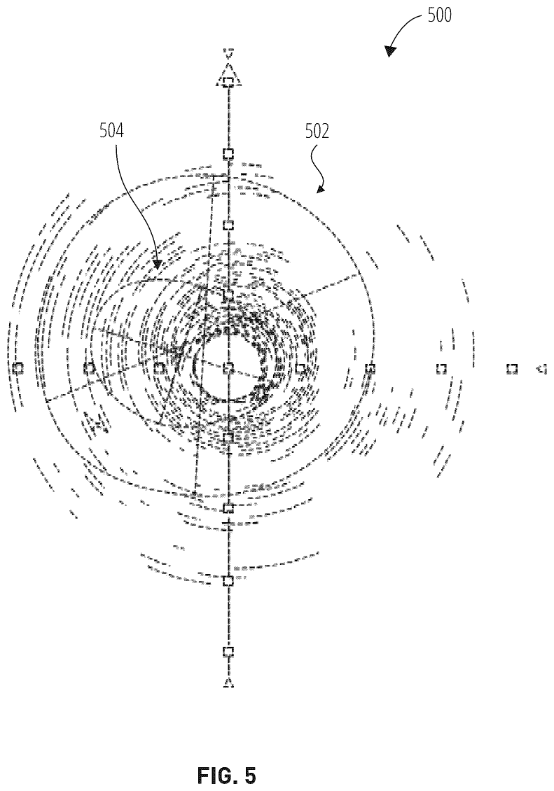

FIG. 5 illustrates an image 500 showing an on-axis view of a frame of series of IVUS image frames 200a (e.g., IVUS image frame 202-1, or the like). As can be seen from the image 500, the structure of the portion of cardiac artery 102 at which the frame is captured is represented. Processor 306 can execute instructions 316 to determine vessel border 502 and/or lumen border 504 from the frame. The determined vessel border 502 and/or lumen border 504 can be stored as vessel and/or lumen assessments 320. Further, processor 306 can execute instructions 316 to derive an area (referred to as the raw area) of the vessel and/or lumen based on the vessel border 502 and/or lumen border 504, respectively. The derived area(s) can be stored as vessel and/or lumen assessments 320. Additionally, with some embodiments, the derived lumen area can be smoothed, and the smoothed vessel and/or lumen area can be stored as vessel and/or lumen assessments 320. For example, processor 306 can execute instructions 316 to filter and/or “smooth” the derived areas using a sample filter (e.g., 21-sample filter, or the like). As another example, processor 306 can execute instructions 316 to filter and/or “smooth” the derived areas using a distance filter (e.g., 1 millimeter (mm), 2 mm, 3 mm, etc.)

Returning to FIG. 4, logic flow 400 can continue to block 408 “identify a second subset of frames of the series of IVUS image frames in which a side branch of the cardiac artery is represented” a second subset of the frames of the series of IVUS image frames in which a side branch or branches (e.g., side branches 104, or the like) are represented are identified. For example, processor 306 can execute instructions 316 to identify the subset of IVUS image frames including a side branch 322 from the series of IVUS image frames 200a based on a side branch detection technique and/or system.

In some embodiments, block 404, block 406, and block 408 of logic flow 400 may be omitted (e.g., implemented as part of another workflow by IVUS navigation system 300, or the like.) In such an example, processor 306 can execute instructions 316 to receive indications of the subset of IVUS image frames including a stent 318, the vessel and/or lumen assessments 320, and the subset of IVUS image frames including a side branch 322, where the subset of IVUS image frames including a stent 318, the vessel and/or lumen assessments 320, and/or the subset of IVUS image frames including a side branch 322 are identified or derived previously (e.g., as part of another workflow).

Continuing to block 410 “identify a distal key frame from the series of IVUS image frames based on the first and second subset of frames, the derived vessel and/or lumen assessments, and key frame marker (KFM) thresholds” a distal key frame from the frames of the series of IVUS image frames can be identified based on the first and second subset of frames, the vessel and/or lumen assessments, and the KFM threshold. For example, processor 306 can execute instructions 316 to identify frames of the series of IVUS image frames 200a within a distance specified by the key frame marker thresholds 324 from the distal edge of the stent defined by subset of IVUS image frames including a stent 318; and identify as the distal key frame 326, a frame from the frames within the threshold distance based on the lumen area and plaque burden of the frame defined by vessel and/or lumen assessments 320 and the locations of side branches proximate to the frame defined by subset of IVUS image frames including a side branch 322. A more detailed example of this is provided with respect to the logic flow 800 and FIG. 8 discussed below.

Continuing to block 412 “identify a proximal key frame from the series of IVUS image frames based on the first and second subset of frames, the derived vessel and/or lumen assessments, and the KFM thresholds” a proximal key frame from the frames of the series of IVUS image frames can be identified based on the first and second subset of frames, the vessel and/or lumen assessments, and the KFM threshold. For example, processor 306 can execute instructions 316 to identify frames of the series of IVUS image frames 200a within a distance specified by the key frame marker thresholds 324 from the proximal edge of the stent defined by subset of IVUS image frames including a stent 318; and identify as the proximal key frame 328, a frame from the frames within the threshold distance based on the lumen area and plaque burden of the frame defined by vessel and/or lumen assessments 320 and the locations of side branches proximate to the frame defined by subset of IVUS image frames including a side branch 322. A more detailed example of this is provided with respect to the logic flow 900 and FIG. 9 discussed below.

Continuing to block 414 “identify a minimum key frame from the series of IVUS image frames based on the distal key frame and the proximal key frame” a minimum key frame from the frames of the series of IVUS image frames can be identified based on the distal key frame and the proximal key frame. For example, processor 306 can execute instructions 316 to identify the frame of the series of IVUS image frames 200a between the distal key frame 326 and proximal key frame 328 with the minimum lumen area. With some embodiments, processor 306 can execute instructions 316 to identify the minimum key frame based on the vessel and/or lumen assessments 320 and select as the minimum key frame, the frame of the series of IVUS images including a stent 318 where the ratio of the lumen border over the vessel border is the lowest.

Continuing to block 416 “generate a graphical user interface comprising indications of the series of IVUS image frames and the distal, proximal, and minimum key frames” a graphical user interface (GUI) comprising indications of the series of IVUs image frames received at block 402 and the key frames identified at blocks 410, 412, and 414 can be generated. For example, processor 306 can execute instructions 316 to generate graphical user interface (GUI) 334 based on the series of IVUS image frames 200a, the distal key frame 326, the proximal key frame 328, and the minimum key frame 330. With some examples, processor 306 can execute instructions 316 to generate key frame marker graphical indications 332 comprising graphical markers to indicate locations of the key frames and generate graphical user interface (GUI) 334 based on the series of IVUS image frames 200a and the key frame marker graphical indications 332.

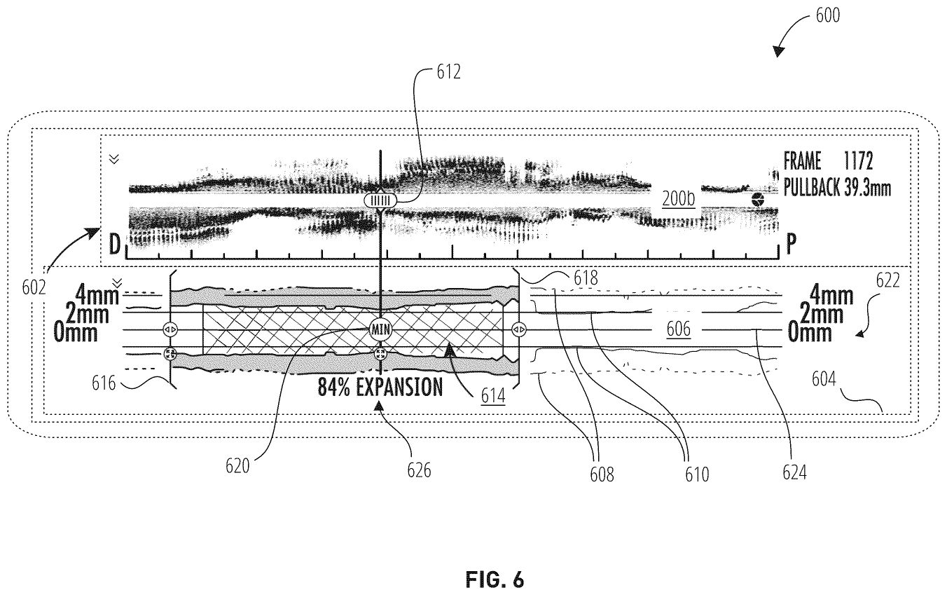

FIG. 6 illustrates a GUI 600, which can be generated according to some embodiments of the present disclosure. For example, GUI 600 can be generated by IVUS navigation system 300 as GUI 334 and displayed on display 304. With some embodiments, responsive to detection of a stent in series of IVUS image frames 200a, processor 306 can execute instructions 316 to generate GUI 600.

GUI 600 can include a longitudinal vessel graphical indication 602 and a longitudinal vessel depiction graphical indication 604. The longitudinal vessel graphical indication 602 can include longitudinal view 200b while longitudinal vessel depiction graphical indication 604 can include a vessel profile 606 of longitudinal view 200b showing vessel borders 608 and lumen borders 610. Further, GUI 600 can include a slider 612, which can be manipulated (e.g., via I/O devices 310, or the like) to move or slide along the frames in series of IVUS image frames 200a depicted by longitudinal view 200b.

Longitudinal vessel depiction graphical indication 604 can further include a depiction of the stent 106 represented in longitudinal view 200b as well as indications of the key frames identified as outlined herein. For example, FIG. 6 depicts GUI 600 with longitudinal vessel depiction graphical indication 604 showing stent 614 (e.g., representative of stent 106 in cardiac artery 102) as well as distal key frame marker 616, proximal key frame marker 618, and minimum key frame marker 620. The distal key frame marker 616, proximal key frame marker 618, and minimum key frame marker 620 can correspond to the KFMs (e.g., distal key frame 326, proximal key frame 328, and minimum key frame 330 identified as outlined herein).

With some embodiments GUI 600 can include a scale 622 measured radially from a longitudinal axis 624 about which the vessel profile 606 is depicted. Processor 306 can execute instructions 316 to shade, color, or otherwise format the graphical visualization (e.g., line weight, solid line, dashed line, dotted line, area shading, area color, area pattern, etc.) to indicate plaque, confidence of border detection, a detected stent, or the like. For example, the area between vessel border 608 and lumen border 610 and between the distal key frame marker 616 and proximal key frame marker 618 can be shaded a different color than the background of GUI 600 to indicate a plaque burden of this region of the cardiac artery 102. Similarly, portions of the lines indicating the vessel border 608 and/or the lumen border 610 can be solid while other portions can be dashed indicating a confidence in the detection of the border for that respective frame of series of IVUS image frames 200a (or section of longitudinal view 200b). Similarly, vessel border 608 and lumen border 610 can be different colors to indicate which is the vessel border and which is the lumen border. With further examples, the stent 614 can be represented as a patterned area, such as, with hatch marks or the like.

Additionally, GUI 600 can include a graphical representation of a stent expansion. For example, GUI 600 includes expansion graphical indication 626 showing (e.g., as a percentage, ratio, or the like) a determined expansion of the stent 106 represented by stent 614. With some examples, the expansion ratio shown in expansion graphical indication 626 can be derived based on the minimum stent area (MSA) divided by the lumen area multiplied by 100 and visualized as a percentage as shown.

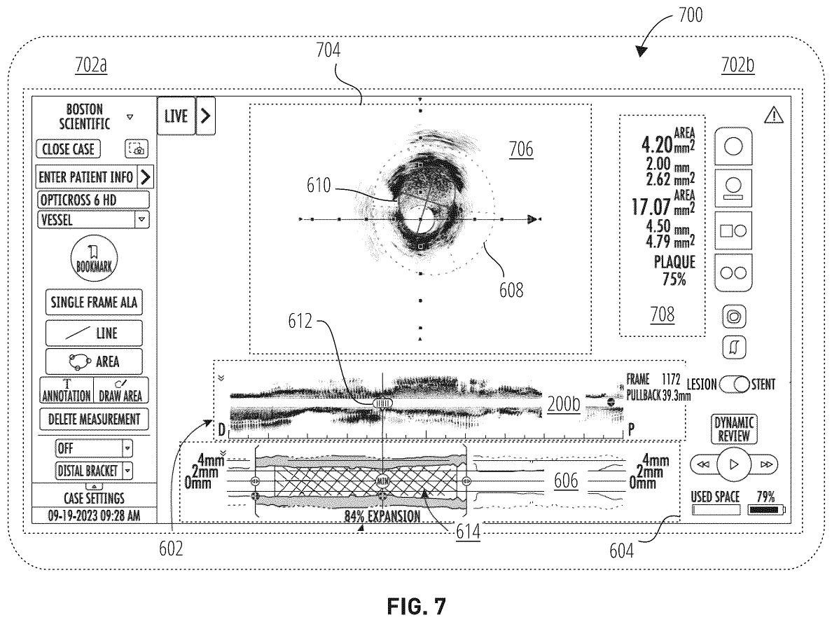

FIG. 7 illustrates a GUI 700, which can be generated according to some embodiments of the present disclosure. For example, GUI 700 can be generated by IVUS navigation system 300 as GUI 334 and displayed on display 304. With some embodiments, responsive to detection of a stent in series of IVUS image frames 200a, processor 306 can execute instructions 316 to generate GUI 700. It is noted that GUI 700 includes some graphical features of GUI 600. As such, GUI 700 is described with reference to GUI 600.

GUI 700 can includes menu 702a and menu 702b disposed on either sides of (or framing) graphical representations of cross-sectional frame graphical indication 704, longitudinal vessel graphical indication 602, and longitudinal vessel depiction graphical indication 604 where longitudinal vessel graphical indication 602 shows longitudinal view 200b and longitudinal vessel depiction graphical indication 604 shows vessel profile 606 and stent 614 as described above with respect to FIG. 6 and GUI 600.

The cross-sectional frame graphical indication 704 can include depictions of on-axis IVUS image frame view 706 corresponding to the frame of series of IVUS image frames 200a in which the slider 612 is positioned. Further, cross-sectional frame graphical indication 704 can include indications of vessel border 608 and/or lumen border 610 as may be derived based on the frame depicted in on-axis IVUS image frame view 706.

In some examples, GUI 700 can include assessment graphical indication 708 showing graphical representations of vessel and/or lumen assessments 320 (e.g., vessel and/or lumen area, plaque burden, etc.

FIG. 8 illustrates a logic flow 800, which can be implemented to identify the distal key frame 326 from series of IVUS image frames 200a. In some examples, logic flow 400 of FIG. 4 can implement logic flow 800 at block 410. Logic flow 800 can begin at block 802. At block 802 “identify the distal edge of the stent” the distal edge of the stent can be identified. For example, processor 306 can execute instructions 316 to identify the stent distal edge 118 in the series of IVUS image frames 200a.

Continuing to decision block 804 “is the distal edge of the stent less than or equal to a first threshold distance from the start of the run?” a determination can be made as to whether the distal edge of the stent is less than or equal to a first threshold distance from the start of the run. As used herein, the term “run” means the set of IVUS image frames, such as, series of IVUS image frames 200a captured while IVUS imaging catheter 108 is pulled back from the distal point 114 to the proximal point 116. Accordingly, the start of the run will be at distal point 114 and the end of the run will be at proximal point 116. For example, processor 306 can execute instructions 316 to determine whether the stent distal edge 118 is within a first threshold distance (e.g., a threshold of key frame marker thresholds 324) from distal point 114. In some examples, the first threshold distance can be 0.5 mm. With some examples, the first threshold distance is 0.25 mm, 0.30 mm, 0.35 mm, 0.40 mm, 0.45 mm, 0.55 mm, 0.60 mm, 0.65 mm, 0.70 mm, or 0.75 mm. With some examples, the first threshold distance is greater than or equal to 0.25 mm and less than or equal to 1 mm.

From decision block 804, logic flow 800 can continue to block 806 or decision block 808. For example, logic flow 800 can continue from decision block 804 to block 806 where a determination is made at decision block 804 that the distal edge of the stent is less than or equal to the first threshold distance from the start of the run while logic flow 800 can continue from decision block 804 to decision block 808 where a determination is made at decision block 804 that the distal edge of the stent is not less than or equal to the first threshold distance from the start of the run.

At block 806 “select the first frame in the run as the distal key frame” the first frame in the run can be selected as the distal key frame. For example, processor 306 can execute instructions 316 to identify as the distal key frame 326, the first frame (e.g., most distal frame, first captured frame, or the like) in series of IVUS image frames 200a. In this example, processor 306 can execute instructions 316 to identify IVUS image frame 202-1 as the distal key frame 326.

At decision block 808 “is the distal edge of the stent greater than the first threshold distance and less than or equal to a second threshold distance from the start of the run?” a determination can be made as to whether the distal edge of the stent is greater than the first threshold distance and less than or equal to a second threshold distance from the start of the run. For example, processor 306 can execute instructions 316 to determine whether the stent distal edge 118 is greater than the first threshold distance (e.g., a first threshold of key frame marker thresholds 324) and less than or equal to a second threshold distance (e.g., another threshold of key frame marker thresholds 324) from distal point 114. In some examples, the second threshold distance can be 5 mm. With some examples, the second threshold distance is 4.0 mm, 4.25 mm, 4.5 mm, 4.75 mm, 5.25 mm, 5.5 mm, 5.75 mm, or 6 mm. With some examples, the second threshold distance is greater than or equal to 4 mm and less than or equal to 6 mm.

From decision block 808, logic flow 800 can continue to block 810 or block 812. For example, logic flow 800 can continue from decision block 808 to block 810 where a determination is made at decision block 808 that the distal edge of the stent is greater than the first threshold distance and less than or equal to a second threshold distance from the start of the run while logic flow 800 can continue from decision block 808 to block 812 where a determination is made at decision block 808 that the distal edge of the stent is not greater than the first threshold distance and less than or equal to a second threshold distance from the start of the run.

At block 810 “select as a subset of frames of the run, frames between the first threshold distance distal to the distal edge of the stent and the start of the run” frames of the series of IVUS image frames (e.g., received at block 402 of logic flow 400 depicted in FIG. 4) that are between the first threshold distance (e.g., 0.5 mm) and the start of the run can be selected as a subset of frames.

At block 812 “select as a subset of frames of the run, frames between the first threshold distance and the second threshold distance distal to the distal edge of the stent” frames of the series of IVUS image frames (e.g., received at block 402 of logic flow 400 depicted in FIG. 4) that are between the first threshold distance (e.g., 0.5 mm) and the second threshold distance (e.g., 5 mm) distal to the stent distal edge 118 of the stent 106 can be selected as a subset of frames. It is noted that block 810 and block 812 are alternatively implemented in logic flow 800, as such, only a single subset of frames is generated.

Logic flow 800 can continue from block 810 and block 812 to decision block 814. At decision block 814 “is there a side branch in the subset of frames?” a determination can be made as to whether there is a side branch represented in a frame or frames of the subset of frames (e.g., frames identified at block 810 or block 812). For example, processor 306 can execute instructions 316 to determine whether there is a side branch (e.g., side branches 104, or the like) represented in the frames of series of IVUS image frames 200a identified as the subset of frames at block 810 or block 812 based on subset of IVUS image frames including a side branch 322.

From decision block 814, logic flow 800 can continue to decision block 816 or block 822. For example, logic flow 800 can continue from decision block 814 to decision block 816 where a determination is made at decision block 814 that there is not a side branch represented in a frame or frames of the subset of frames while logic flow 800 can continue from decision block 814 to block 822 where a determination is made at decision block 814 that there is a side branch represented in a frame or frames of the subset of frames.

At decision block 816 “frame(s) within the subset with a smooth and a raw plaque burden less than or equal to 50%?” a determination is made as to whether there are frames in the subset of frames where the smooth plaque burden and the raw plaque burden are less than or equal to a threshold value. For example, processor 306 can execute instructions 316 to determine whether there are frames in the frames of series of IVUS image frames 200a identified as the subset of frames at block 810 or block 812 where the plaque burden (e.g., smooth, raw, smooth and raw, or the like) is less than or equal to a threshold value. In some embodiments, processor 306 can execute instructions 316 to determine whether both the smooth and raw plaque burden as defined in vessel and/or lumen assessments 320 are less than or equal to a threshold value. With some embodiments, processor 306 can execute instructions 316 to determine whether the smooth plaque burden as defined in vessel and/or lumen assessments 320 is less than or equal to a first threshold value and whether the raw plaque burden as defined in vessel and/or lumen assessments 320 is less than or equal to a second, different threshold value. With some embodiments, processor 306 can execute instructions 316 to determine whether the smooth and/or raw plaque burden as defined in vessel and/or lumen assessments 320 are less than or equal to a first and/or second threshold value.

In some examples, the threshold value or the first and/or second threshold value can be 50%. With some examples, the first and second threshold values are different. With some examples, the threshold value or the first and/or second threshold values are 40%, 45%, 55%, or 60%. With some examples, the threshold value or the first and/or second threshold values is greater than or equal to 40% and less than or equal to 60%.

From decision block 816, logic flow 800 can continue to block 818 or block 820. For example, logic flow 800 can continue from decision block 816 to block 818 where a determination is made at decision block 816 that there are not frames in the subset of frames where the smooth plaque burden and the raw plaque burden are less than or equal to a threshold value while logic flow 800 can continue from decision block 816 to block 820 where a determination is made at decision block 816 that there are frames in the subset of frames where the smooth plaque burden and the raw plaque burden are less than or equal to a threshold value.

At block 818 “select the frame from the subset of frames that is most distal to the distal edge of the stent as the distal key frame” the frame from the subset of frames most distal to the distal edge of the stent (e.g., the first frame or the frame that is the second threshold distance distal to the distal edge of the stent) can be selected as the distal key frame. For example, processor 306 can execute instructions 316 to designate the frame in the frames of series of IVUS image frames 200a identified as the subset of frames at block 810 or block 812 that is most distal to the stent distal edge 118 as the distal key frame 326.

At block 820 “select the frame from the subset of frames with the largest lumen area having a smooth/raw plaque burden less than the threshold value that is closest to the distal edge of the stent as the distal key frame” the most proximal frame from the subset of frames having the largest lumen area and having a smooth and/or raw plaque burden less than or equal to the threshold value can be selected as the distal key frame. For example, processor 306 can execute instructions 316 to designate the most proximal frame in the frames of series of IVUS image frames 200a identified as the subset of frames at block 810 or block 812 that has the largest lumen area and has a smooth and/or raw plaque burden less than or equal to the threshold value (e.g., 50%, or the like) as the distal key frame 326. As noted above, vessel and/or lumen assessments 320 can define the lumen area for each frame of series of IVUS image frames 200a.

At block 822 “select the frame that is the first threshold distance distal to the distal edge of the stent as the distal key frame” the frame from the subset of frames that is the first threshold distance from the distal edge of the stent (e.g., the frame of the subset of frames closest to the distal edge of the stent) can be selected as the distal key frame. For example, processor 306 can execute instructions 316 to designate the frame in the frames of series of IVUS image frames 200a identified as the subset of frames at block 810 or block 812 that is closest to the stent distal edge 118 (e.g., that is the first threshold distance from) as the distal key frame 326.

From block 806, block 818, block 820, and block 822, the logic flow 800 can end.

FIG. 9 illustrates a logic flow 900, which can be implemented to identify the proximal key frame 328 from series of IVUS image frames 200a. In some examples, logic flow 400 of FIG. 4 can implement logic flow 900 at block 412. Logic flow 900 can begin at block 902. At block 902 “identify the proximal edge of the stent” the proximal edge of the stent can be identified. For example, processor 306 can execute instructions 316 to identify the stent proximal edge 120 in the series of IVUS image frames 200a.

Continuing to decision block 904 “is the proximal edge of the stent less than or equal to a first threshold distance from the end of the run?” a determination can be made as to whether the proximal edge of the stent is less than or equal to a first threshold distance from the end of the run. As used herein, the term “run” means the set of IVUS image frames, such as, series of IVUS image frames 200a captured while IVUS imaging catheter 108 is pulled back from the distal point 114 to the proximal point 116. Accordingly, the start of the run will be at distal point 114 and the end of the run will be at proximal point 116. For example, processor 306 can execute instructions 316 to determine whether the stent proximal edge 120 is within a first threshold distance (e.g., a threshold of key frame marker thresholds 324) from proximal point 116. In some examples, the first threshold distance can be 0.5 mm. With some examples, the first threshold distance is 0.25 mm, 0.30 mm, 0.35 mm, 0.40 mm, 0.45 mm, 0.55 mm, 0.60 mm, 0.65 mm, 0.70 mm, or 0.75 mm. With some examples, the first threshold distance is greater than or equal to 0.25 mm and less than or equal to 1 mm.

From decision block 904, logic flow 900 can continue to block 906 or decision block 908. For example, logic flow 900 can continue from decision block 904 to block 906 where a determination is made at decision block 904 that the proximal edge of the stent is less than or equal to the first threshold distance from the end of the run while logic flow 900 can continue from decision block 904 to decision block 908 where a determination is made at decision block 904 that the proximal edge of the stent is not less than or equal to the first threshold distance from the end of the run.

At block 906 “select the last frame in the run as the proximal key frame” the last frame in the run can be selected as the proximal key frame. For example, processor 306 can execute instructions 316 to identify as the proximal key frame 328, the last frame (e.g., most proximal frame, last captured frame, or the like) in series of IVUS image frames 200a. In this example, processor 306 can execute instructions 316 to identify IVUS image frame 202-n as the proximal key frame 328.

At decision block 908 “is the proximal edge of the stent greater than the first threshold distance and less than or equal to a second threshold distance from the end of the run?” a determination can be made as to whether the proximal edge of the stent is greater than the first threshold distance and less than or equal to a second threshold distance from the end of the run. For example, processor 306 can execute instructions 316 to determine whether the stent proximal edge 120 is greater than the first threshold distance (e.g., a first threshold of key frame marker thresholds 324) and less than or equal to a second threshold distance e.g., another threshold of key frame marker thresholds 324) from proximal point 116. In some examples, the second threshold distance can be 5 mm. With some examples, the second threshold distance is 4.0 mm, 4.25 mm, 4.5 mm, 4.75 mm, 5.25 mm, 5.5 mm, 5.75 mm, or 6 mm. With some examples, the second threshold distance is greater than or equal to 4 mm and less than or equal to 6 mm.

From decision block 908, logic flow 900 can continue to block 910 or block 912. For example, logic flow 900 can continue from decision block 908 to block 910 where a determination is made at decision block 908 that the proximal edge of the stent is greater than the first threshold distance and less than or equal to a second threshold distance from the end of the run while logic flow 900 can continue from decision block 908 to block 912 where a determination is made at decision block 908 that the proximal edge of the stent is not greater than the first threshold distance and less than or equal to a second threshold distance from the end of the run.

At block 910 “select as a subset of frames of the run, frames between the first threshold distance proximal to the proximal edge of the stent and the end of the run” frames of the series of IVUS image frames (e.g., received at block 402 of logic flow 400 depicted in FIG. 4) that are between the first threshold distance (e.g., 0.5 mm) and the end of the run can be selected as a subset of frames.

At block 912 “select as a subset of frames of the run, frames between the first threshold distance and the second threshold distance proximal to the proximal edge of the stent” frames of the series of IVUS image frames (e.g., received at block 402 of logic flow 400 depicted in FIG. 4) that are between the first threshold distance (e.g., 0.5 mm) and the second threshold distance (e.g., 5 mm) proximal to the stent proximal edge 120 of the stent 106 can be selected as a subset of frames. It is noted that block 910 and block 912 are alternatively implemented in logic flow 900, as such, only a single subset of frames is generated.

Logic flow 900 can continue from block 910 and block 912 to decision block 914. At decision block 914 “is there a side branch in the subset of frames?” a determination can be made as to whether there is a side branch represented in a frame or frames of the subset of frames (e.g., frames identified at block 910 or block 912). For example, processor 306 can execute instructions 316 to determine whether there is a side branch (e.g., side branches 104, or the like) represented in the frames of series of IVUS image frames 200a identified as the subset of frames at block 910 or block 912 based on subset of IVUS image frames including a side branch 322.

From decision block 914, logic flow 900 can continue to decision block 916 or block 922. For example, logic flow 900 can continue from decision block 914 to decision block 916 where a determination is made at decision block 914 that there is not a side branch represented in a frame or frames of the subset of frames while logic flow 900 can continue from decision block 914 to block 922 where a determination is made at decision block 914 that there is a side branch represented in a frame or frames of the subset of frames.

At decision block 916 “frame(s) within the subset with a smooth and a raw plaque burden less than or equal to 50%?” a determination is made as to whether there are frames in the subset of frames where the smooth plaque burden and the raw plaque burden are less than or equal to a threshold value. For example, processor 306 can execute instructions 316 to determine whether there are frames in the frames of series of IVUS image frames 200a identified as the subset of frames at block 910 or block 912 where the plaque burden (e.g., smooth, raw, smooth and raw, or the like) is less than or equal to a threshold value. In some embodiments, processor 306 can execute instructions 316 to determine whether both the smooth and raw plaque burden as defined in vessel and/or lumen assessments 320 are less than or equal to a threshold value. With some embodiments, processor 306 can execute instructions 316 to determine whether the smooth plaque burden as defined in vessel and/or lumen assessments 320 is less than or equal to a first threshold value and whether the raw plaque burden as defined in vessel and/or lumen assessments 320 is less than or equal to a second, different threshold value. With some embodiments, processor 306 can execute instructions 316 to determine whether the smooth and/or raw plaque burden as defined in vessel and/or lumen assessments 320 are less than or equal to a first and/or second threshold value.

In some examples, the threshold value or the first and/or second threshold value can be 50%. With some examples, the first and second threshold values are different. With some examples, the threshold value or the first and/or second threshold values are 40%, 45%, 55%, or 60%. With some examples, the threshold value or the first and/or second threshold values is greater than or equal to 40% and less than or equal to 60%.

From decision block 916, logic flow 900 can continue to block 918 or block 920. For example, logic flow 900 can continue from decision block 916 to block 918 where a determination is made at decision block 916 that there are not frames in the subset of frames where the smooth plaque burden and the raw plaque burden are less than or equal to a threshold value while logic flow 900 can continue from decision block 916 to block 920 where a determination is made at decision block 916 that there are frames in the subset of frames where the smooth plaque burden and the raw plaque burden are less than or equal to a threshold value.

At block 918 “select the frame from the subset of frames that is most proximal to the proximal edge of the stent as the proximal key frame” the frame from the subset of frames most proximal to the proximal edge of the stent (e.g., the last frame or the frame that is the second threshold distance proximal to the proximal edge of the stent) can be selected as the proximal key frame. For example, processor 306 can execute instructions 316 to designate the frame in the frames of series of IVUS image frames 200a identified as the subset of frames at block 910 or block 912 that is most proximal to the stent proximal edge 120 as the proximal key frame 328.

At block 920 “select the frame from the subset of frames with the largest lumen area having a smooth/raw plaque burden less than the threshold value that is closest to the proximal edge of the stent as the proximal key frame” the most distal frame from the subset of frames having the largest lumen area and having a smooth and/or raw plaque burden less than or equal to the threshold value can be selected as the proximal key frame. For example, processor 306 can execute instructions 316 to designate the most distal frame in the frames of series of IVUS image frames 200a identified as the subset of frames at block 910 or block 912 that has the largest lumen area and has a smooth and/or raw plaque burden less than or equal to the threshold value (e.g., 50%, or the like) as the proximal key frame 328. As noted above, vessel and/or lumen assessments 320 can define the lumen area for each frame of series of IVUS image frames 200a.

At block 922 “select the frame that is the first threshold distance proximal to the proximal edge of the stent as the proximal key frame” the frame from the subset of frames that is the first threshold distance from the proximal edge of the stent (e.g., the frame of the subset of frames closest to the proximal edge of the stent) can be selected as the proximal key frame. For example, processor 306 can execute instructions 316 to designate the frame in the frames of series of IVUS image frames 200a identified as the subset of frames at block 910 or block 912 that is closest to the stent proximal edge 120 (e.g., that is the first threshold distance from) as the proximal key frame 328.

From block 906, block 918, block 920, and block 922, the logic flow 900 can end.

It is noted that often, an IVUS navigation system (e.g., IVUS navigation system 300, or the like) can implemented both logic flows 800 and 900 as part of the same overall logic flow to identify distal and proximal key frames as outlined herein. For example, IVUS navigation system 300 can implement logic flow 400 to identify key frames as outlined above. As part of logic flow 400, IVUS navigation system can implement logic flow 800 (e.g., at block 410) and logic flow 900 (e.g., at block 412). Where both logic flow 800 and 900 are implemented, the threshold distances used to identify each distal and proximal key frame as detailed in respective descriptions of logic flow 800 and 900 may be the same or they may be different. For example, at decision block 804 of logic flow 800, processor 306 can execute instructions 316 to determine whether the stent distal edge 118 is within a first threshold distance (e.g., a threshold of key frame marker thresholds 324) from distal point 114. Likewise, at decision block 904 of logic flow 900, processor 306 can execute instructions 316 to determine whether the stent proximal edge 120 is within a second threshold distance (e.g., a threshold of key frame marker thresholds 324) from proximal point 116. In such an example, the first threshold (e.g., used at block 804) may be the same or different from the second threshold (e.g., used at block 904).

Continuing with this example, at decision block 808 of logic flow 800, processor 306 can execute instructions 316 to determine whether the stent distal edge 118 is greater than the first threshold distance and less than or equal to a third threshold distance (e.g., another threshold of key frame marker thresholds 324) from distal point 114. Likewise, at decision block 908 of logic flow 900, processor 306 can execute instructions 316 to determine whether the stent proximal edge 120 is greater than the second threshold distance and less than or equal to a fourth threshold distance (e.g., another threshold of key frame marker thresholds 324) from proximal point 116. In such an example, the third threshold (e.g., used at block 808) may be the same or different from the fourth threshold (e.g., used at block 908).

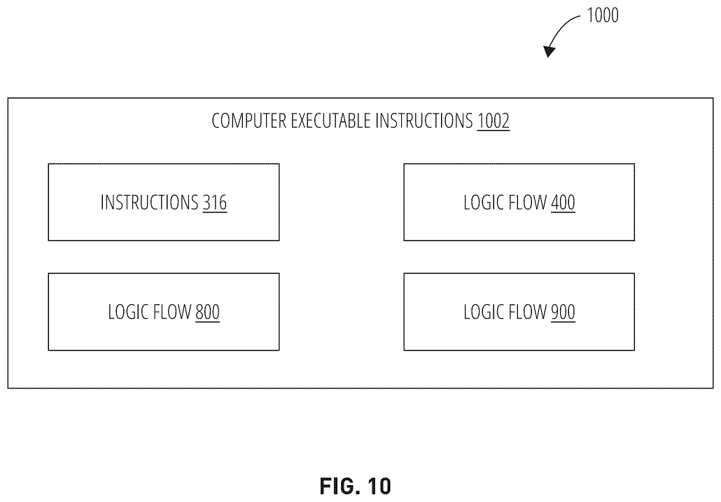

FIG. 10 illustrates computer-readable storage medium 1000. Computer-readable storage medium 1000 may comprise any non-transitory computer-readable storage medium or machine-readable storage medium, such as an optical, magnetic or semiconductor storage medium. In various embodiments, computer-readable storage medium 1000 may comprise an article of manufacture. In some embodiments, computer-readable storage medium 1000 may store computer executable instructions 1002 with which circuitry (e.g., image acquisition circuitry 126, processor 306, IVUS imaging system acquisition circuitry 314, and the like) can execute. For example, computer executable instructions 1002 can include instructions to implement operations described with respect to instructions 316, logic flow 400, logic flow 800, and/or logic flow 900. Examples of computer-readable storage medium 1000 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1002 may include any suitable type of code, such as source code, compiled code, interpreted code, executable code, static code, dynamic code, object-oriented code, visual code, and the like.

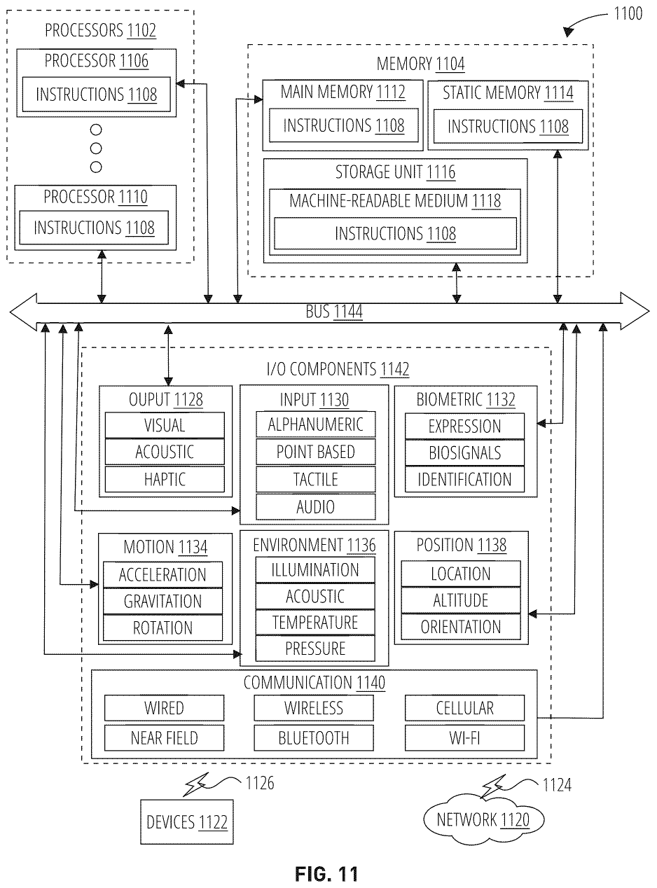

FIG. 11 illustrates a diagrammatic representation of a machine 1100 in the form of a computer system within which a set of instructions may be executed for causing the machine to perform any one or more of the methodologies discussed herein. More specifically, FIG. 11 shows a diagrammatic representation of the machine 1100 in the example form of a computer system, within which instructions 1108 (e.g., software, a program, an application, an applet, an app, or other executable code) for causing the machine 1100 to perform any one or more of the methodologies discussed herein may be executed. For example, the instructions 1108 may cause the machine 1100 to execute logic flow 400 of FIG. 4, logic flow 800 of FIG. 8, logic flow 900 of FIG. 9, or the like. More generally, the instructions 1108 may cause the machine 1100 to automatically determine key frames during a post-PCI procedure. It is noted that the present disclosure provides specific and discrete implementations of identifying key frames (e.g., distal key frame 326, proximal key frame 328, and/or minimum key frame 330) which is a significant improvement over the prior art. In particular, the present disclosure provides an improvement to computing technology in that the distal and proximal key frames are identified based on a location of a stent (e.g., stent 106, or the like).

The instructions 1108 transform the general, non-programmed machine 1100 into a particular machine 1100 programmed to carry out the described and illustrated functions in a specific manner. In alternative embodiments, the machine 1100 operates as a standalone device or may be coupled (e.g., networked) to other machines. In a networked deployment, the machine 1100 may operate in the capacity of a server machine or a client machine in a server-client network environment, or as a peer machine in a peer-to-peer (or distributed) network environment. The machine 1100 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1108, sequentially or otherwise, that specify actions to be taken by the machine 1100. Further, while a single machine 1100 is illustrated, the term “machine” shall also be taken to include a collection of machines 1100 that individually or jointly execute the instructions 1108 to perform any one or more of the methodologies discussed herein.

The machine 1100 may include processors 1102, memory 1104, and I/O components 1142, which may be configured to communicate with each other such as via a bus 1144. In an example embodiment, the processors 1102 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1106 and a processor 1110 that may execute the instructions 1108. The term “processor” is intended to include multi-core processors that may comprise two or more independent processors (sometimes referred to as “cores”) that may execute instructions contemporaneously. Although FIG. 11 shows multiple processors 1102, the machine 1100 may include a single processor with a single core, a single processor with multiple cores (e.g., a multi-core processor), multiple processors with a single core, multiple processors with multiples cores, or any combination thereof.

The memory 1104 may include a main memory 1112, a static memory 1114, and a storage unit 1116, both accessible to the processors 1102 such as via the bus 1144. The main memory 1104, the static memory 1114, and storage unit 1116 store the instructions 1108 embodying any one or more of the methodologies or functions described herein. The instructions 1108 may also reside, completely or partially, within the main memory 1112, within the static memory 1114, within machine-readable medium 1118 within the storage unit 1116, within at least one of the processors 1102 (e.g., within the processor's cache memory), or any suitable combination thereof, during execution thereof by the machine 1100.

The I/O components 1142 may include a wide variety of components to receive input, provide output, produce output, transmit information, exchange information, capture measurements, and so on. The specific I/O components 1142 that are included in a particular machine will depend on the type of machine. For example, portable machines such as mobile phones will likely include a touch input device or other such input mechanisms, while a headless server machine will likely not include such a touch input device. It will be appreciated that the I/O components 1142 may include many other components that are not shown in FIG. 11. The I/O components 1142 are grouped according to functionality merely for simplifying the following discussion and the grouping is in no way limiting. In various example embodiments, the I/O components 1142 may include output components 1128 and input components 1130. The output components 1128 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1130 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1142 may include biometric components 1132, motion components 1134, environmental components 1136, or position components 1138, among a wide array of other components. For example, the biometric components 1132 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1134 may include acceleration sensor components (e.g., accelerometer), gravitation sensor components, rotation sensor components (e.g., gyroscope), and so forth. The environmental components 1136 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1138 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1142 may include communication components 1140 operable to couple the machine 1100 to a network 1120 or devices 1122 via a coupling 1124 and a coupling 1126, respectively. For example, the communication components 1140 may include a network interface component or another suitable device to interface with the network 1120. In further examples, the communication components 1140 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1122 may be another machine or any of a wide variety of peripheral devices (e.g., a peripheral device coupled via a USB).

Moreover, the communication components 1140 may detect identifiers or include components operable to detect identifiers. For example, the communication components 1140 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1140, such as location via Internet Protocol (IP) geolocation, location via Wi-Fi® signal triangulation, location via detecting an NFC beacon signal that may indicate a particular location, and so forth.

The various memories (i.e., memory 1104, main memory 1112, static memory 1114, and/or memory of the processors 1102) and/or storage unit 1116 may store one or more sets of instructions and data structures (e.g., software) embodying or utilized by any one or more of the methodologies or functions described herein. These instructions (e.g., the instructions 1108), when executed by processors 1102, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1120 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 1120 or a portion of the network 1120 may include a wireless or cellular network, and the coupling 1124 may be a Code Division Multiple Access (CDMA) connection, a Global System for Mobile communications (GSM) connection, or another type of cellular or wireless coupling. In this example, the coupling 1124 may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1108 may be transmitted or received over the network 1120 using a transmission medium via a network interface device (e.g., a network interface component included in the communication components 1140) and utilizing any one of several well-known transfer protocols (e.g., hypertext transfer protocol (HTTP)). Similarly, the instructions 1108 may be transmitted or received using a transmission medium via the coupling 1126 (e.g., a peer-to-peer coupling) to the devices 1122. The terms “transmission medium” and “signal medium” mean the same thing and may be used interchangeably in this disclosure. The terms “transmission medium” and “signal medium” shall be taken to include any intangible medium that can store, encoding, or carrying the instructions 1108 for execution by the machine 1100, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. An intravascular image navigation system, comprising:
a processor coupled; and
a memory storage device coupled to the processor, the memory storage device comprising instructions executable by the processor, which instructions when executed cause the intravascular image navigation system to:
receive a series of intravascular image (IVI) frames captured along a section of a cardiac artery of a patient where a stent is disposed in the section of the cardiac artery;
identify an edge of the stent from the series of IVI frames;
determine whether a location of the edge of the stent is less than or equal to a first threshold distance from an end of the section of the cardiac artery; and
identify a frame of the series of IVI frames captured at the end of the section of the cardiac artery as a key frame if the location of the edge of the stent is less than or equal to the first threshold distance from the end of the section of the cardiac artery; or
identify a frame of one or more first frames of the series of IVI frames as the key frame based on a lumen area and a plaque burden for each of the one or more first frames if the location of the edge of the stent is not less than or equal to the first threshold distance from the end of the section of the cardiac artery,
wherein the one or more first frames are frames captured between the end of the section of the cardiac artery and the edge of the stent.

2. The intravascular image navigation system of claim 1, the edge of the stent is a distal edge, the end of the section of the cardiac artery is a distal end, the key frame is a distal key frame, and the memory storage device further comprising instructions executable by the processor, which instructions when executed cause the intravascular image navigation system to:
identify a proximal edge of the stent from the series of IVI frames;
determine whether a location of the proximal edge of the stent is less than or equal to a second threshold distance from a proximal end of the section of the cardiac artery; and
identify another frame of the series of IVI frames captured at the proximal end of the section of the cardiac artery as a proximal key frame if the location of the proximal edge of the stent is less than or equal to the second threshold distance from the proximal end of the section of the cardiac artery; or
identify another frame of one or more second frames of the series of IVI frames as the proximal key frame based on a lumen area and a plaque burden for each of the one or more second frames if the location of the proximal edge of the stent is not less than or equal to the second threshold distance from the proximal end of the section of the cardiac artery,
wherein the one or more second frames are frames captured between the proximal end of the section of the cardiac artery and the proximal edge of the stent.

3. The intravascular image navigation system of claim 1, wherein the first threshold distance and/or the second threshold distance are 0.5 millimeters (mm) or wherein the first threshold distance and/or the second threshold distance are between 0.25 mm and 0.75 mm.

4. The intravascular image navigation system of claim 1, the memory storage device further comprising instructions executable by the processor, which instructions when executed cause the intravascular image navigation system to identify a first subset of frames of the series of IVI frames based on an automatic stent detection process, wherein the first subset of frames comprises an indication of the stent.

5. The intravascular image navigation system of claim 4, the memory storage device further comprising instructions executable by the processor, which instructions when executed cause the intravascular image navigation system to identify the edge of the stent based on the first subset of frames.

6. The intravascular image navigation system of claim 4, the memory storage device further comprising instructions executable by the processor, which instructions when executed cause the intravascular image navigation system to identify a second subset of frames of the series of IVI frames based on an automatic side branch detection process, wherein the second subset of frames comprises an indication of one or more side branches.

7. The intravascular image navigation system of claim 6, the memory storage device further comprising instructions executable by the processor, which instructions when executed cause the intravascular image navigation system to:
determine whether the one or more first frames of the series of IVI frames comprises indications of the one or more side branches based on the second subset of frames; and
identify the frame of the one or more first frames located distal to the location of the distal edge of the stent by the first threshold distance as the distal key frame if the one or more first frames of the series of IVI frames comprises indications of the one or more side branches.

8. The intravascular image navigation system of claim 4, the memory storage device further comprising instructions executable by the processor, which instructions when executed cause the intravascular image navigation system to:
determine whether the one or more second frames of the series of IVI frames comprises indications of the one or more side branches based on the second subset of frames; and
identify the frame of the one or more second frames located proximal to the location of the proximal edge of the stent by the second threshold distance as the proximal key frame if the one or more second frames of the series of IVI frames comprises indications of the one or more side branches.

9. The intravascular image navigation system of claim 1, the memory storage device further comprising instructions executable by the processor, which instructions when executed cause the intravascular image navigation system to derive the lumen area and the plaque burden for each frame of the series of IVI frames based on a lumen assessment process.

10. The intravascular image navigation system of claim 1, wherein the series of IVI frames and intravascular ultrasound (IVUS) frames.

11. At least one non-transitory machine readable storage devices, comprising instructions that in response to being executed by a processor of an intravascular imaging (IVI) system cause the IVI system to:
receive a series of intravascular image (IVI) frames captured along a section of a cardiac artery of a patient where a stent is disposed in the section of the cardiac artery;
identify an edge of the stent from the series of IVI frames;
determine whether a location of the edge of the stent is less than or equal to a first threshold distance from an end of the section of the cardiac artery; and
identify a frame of the series of IVI frames captured at the end of the section of the cardiac artery as a key frame if the location of the edge of the stent is less than or equal to the first threshold distance from the end of the section of the cardiac artery; or
identify a frame of one or more first frames of the series of IVI frames as the key frame based on a lumen area and a plaque burden for each of the one or more first frames if the location of the edge of the stent is not less than or equal to the first threshold distance from the end of the section of the cardiac artery,
wherein the one or more first frames are frames captured between the end of the section of the cardiac artery and the edge of the stent.

12. The at least one non-transitory machine readable storage devices of claim 11, the edge of the stent is a distal edge, the end of the section of the cardiac artery is a distal end, the key frame is a distal key frame, and the storage devices further comprise instructions that in response to being executed by the processor of the IVI system cause the IVI system to:
identify a proximal edge of the stent from the series of IVI frames;
determine whether a location of the proximal edge of the stent is less than or equal to a second threshold distance from a proximal end of the section of the cardiac artery; and
identify another frame of the series of IVI frames captured at the proximal end of the section of the cardiac artery as a proximal key frame if the location of the proximal edge of the stent is less than or equal to the second threshold distance from the proximal end of the section of the cardiac artery; or
identify another frame of one or more second frames of the series of IVI frames as the proximal key frame based on a lumen area and a plaque burden for each of the one or more second frames if the location of the proximal edge of the stent is not less than or equal to the second threshold distance from the proximal end of the section of the cardiac artery,
wherein the one or more second frames are frames captured between the proximal end of the section of the cardiac artery and the proximal edge of the stent.

13. The at least one non-transitory machine readable storage devices of claim 12, further comprise instructions that in response to being executed by the processor of the IVI system cause the IVI system to derive the lumen area and the plaque burden for each frame of the series of IVI frames based on a lumen assessment process.

14. The at least one non-transitory machine readable storage devices of claim 12, wherein the lumen area is a raw lumen area and wherein the at least one non-transitory machine readable storage devices further comprise instructions that in response to being executed by the processor of the IVI system cause the IVI system to:
derive a smoothed lumen area from each of the raw lumen areas;
determine whether any one of the one or more first frames have a plaque burden less than or equal to a plaque burden threshold; and
determine whether any one of the one or more second frames have a plaque burden less than or equal to a plaque burden threshold;
identify the frame of the one or more first frames furthest from the distal edge of the stent as the distal key frame based on a determination that not one of the one or more first frames have a plaque burden less than or equal to the plaque burden threshold; or
identify the frame of the one or more first frames closest to the distal edge of the stent that has (i) the largest raw lumen area and the largest smoothed lumen area and (ii) a plaque burden less than or equal to the plaque burden threshold as the distal key frame; and
identify the frame of the one or more second frames furthest from the proximal edge of the stent as the proximal key frame based on a determination that not one of the one or more second frames have a plaque burden less than or equal to the plaque burden threshold; or
identify the frame of the one or more second frames closest to the proximal edge of the stent that has (i) the largest raw lumen area and the largest smoothed lumen area and (ii) a plaque burden less than or equal to the plaque burden threshold as the proximal key frame.

15. The at least one non-transitory machine readable storage devices of claim 29, wherein the plaque burden threshold is 50% or between 40% and 60%.

16. The at least one non-transitory machine readable storage devices of claim 12, further comprising instructions that in response to being executed by the processor of the IVI system cause the IVI system to:
generate one or more graphical indications representing the series of IVI frames, the distal key frame, and the proximal key frame; and
send one or more information elements to a display to cause the display to display the one or more graphical indications as part of a graphical user interface.

17. A computer-implemented method for an intravascular image navigation system, comprising:
receiving, at a processor, a series of intravascular image (IVI) frames captured along a section of a cardiac artery of a patient where a stent is disposed in the section of the cardiac artery;
identifying, by the processor, a distal edge of the stent from the series of IVI frames;
identifying, by the processor, a proximal edge of the stent from the series of IVI frames;
determining, by the processor, whether a location of the distal edge of the stent is less than or equal to a first threshold distance from the distal end of the section of the cardiac artery;
determining, by the processor, whether a location of the proximal edge of the stent is less than or equal to a second threshold distance from the proximal end of the section of the cardiac artery; and
identifying, by the processor, the most distal frame of the series of IVI frames as a distal key frame based on a determination that the location of the distal edge of the stent is less than or equal to the first threshold distance from the distal end of the section of the cardiac artery; or identifying, by the processor, a frame of one or more first frames of the series of IVI frames as the distal key frame based on a lumen area and a plaque burden for each of the one or more first frames, where the one or more first frames are located distal to the location of the distal edge of the stent greater than or equal to the first threshold distance and less than or equal to a third threshold distance; and
identifying, by the processor, the most proximal frame of the series of IVI frames as a proximal key frame based on a determination that the location of the proximal edge of the stent is less than or equal to the second threshold distance from the proximal end of the section of the cardiac artery; or
identifying, by the processor, a frame of one or more second frames of the series of IVI frames as the proximal key frame based on a lumen area and a plaque burden for each of the one or more second frames, wherein the one or more second frames are located proximal to the location of the proximal edge of the stent greater than or equal to the second threshold distance and less than or equal to a fourth threshold distance.

18. The computer-implemented method of claim 17, wherein the first threshold distance and/or the second threshold distance are 0.5 millimeters (mm) or wherein the first threshold distance and/or the second threshold distance are between 0.25 mm and 0.75 mm; and wherein the third threshold distance and/or the fourth threshold distance are 5.0 millimeters (mm) or wherein the third threshold distance and/or the fourth threshold distance are between 4.0 mm and 6.0 mm.

19. The computer-implemented method of claim 17, further comprising:
identifying, by the processor, a first subset of frames of the series of IVI frames based on an automatic stent detection process, wherein the first subset of frames comprises an indication of the stent;
identifying, by the processor, the distal edge of the stent and the proximal edge of the stent based on the first subset of frames;
identifying, by the processor, a second subset of frames of the series of IVI frames based on an automatic side branch detection process, wherein the second subset of frames comprises an indication of one or more side branches;
determining, by the processor, whether the one or more first frames of the series of IVI frames comprises indications of the one or more side branches based on the second subset of frames;
identifying, by the processor, the frame of the one or more first frames located distal to the location of the distal edge of the stent the first threshold distance as the distal key frame based on a determination the one or more first frames of the series of IVI frames comprises indications of the one or more side branches;
determining, by the processor, whether the one or more second frames of the series of IVI frames comprises indications of the one or more side branches based on the second subset of frames; and
identifying, by the processor, the frame of the one or more second frames located proximal to the location of the proximal edge of the stent the second threshold distance as the proximal key frame based on a determination the one or more second frames of the series of IVI frames comprises indications of the one or more side branches.

20. The computer-implemented method of claim 17, further comprising:
deriving, by the processor, the lumen area and the plaque burden for at least each of the one or more first frames and the one or more second frames based on a lumen assessment process, wherein the lumen area is a raw lumen area;
deriving, by the processor, a smoothed lumen area from each of the raw lumen areas;
determining, by the processor, whether any one of the one or more first frames have a plaque burden less than or equal to a plaque burden threshold; and
determining, by the processor, whether any one of the one or more second frames have a plaque burden less than or equal to a plaque burden threshold;
identifying, by the processor, the frame of the one or more first frames furthest from the distal edge of the stent as the distal key frame based on a determination that not one of the one or more first frames have a plaque burden less than or equal to the plaque burden threshold; or
identifying, by the processor, the frame of the one or more first frames closest to the distal edge of the stent that has (i) the largest raw lumen area and the largest smoothed lumen area and (ii) a plaque burden less than or equal to the plaque burden threshold as the distal key frame; and
identifying, by the processor, the frame of the one or more second frames furthest from the proximal edge of the stent as the proximal key frame based on a determination that not one of the one or more second frames have a plaque burden less than or equal to the plaque burden threshold; or
identifying, by the processor, the frame of the one or more second frames closest to the proximal edge of the stent that has (i) the largest raw lumen area and the largest smoothed lumen area and (ii) a plaque burden less than or equal to the plaque burden threshold as the proximal key frame.
